(19) United States
(12) Patent Application Publication
(10) Pub. No.: US 2009/0028818 A1
(43) Pub. Date: Jan. 29, 2009

(54) Heterocyclic antiviral compounds

(75) Inventors: Stephen Deems Gabriel, San Mateo, CA (US); Remy Lemoine, San Francisco, CA (US); David Mark Rotstein, Sunnyvale, CA (US); Jutta Wanner, Mountain View, CA (US)

Correspondence Address: ROCHE PALO ALTO LLC;PATENT LAW DEPT. M/S A2-250, 3431 HILLVIEW AVENUE, PALO ALTO, CA, 94304, US

(73) Assignee: Roche Palo Alto LLC

(21) Appl. No.: 12/220,485

(22) Filed: Jul. 24, 2008

Related U.S. Application Data
(60) Provisional application No. 60/961,784, filed on Jul. 24, 2007.

(51) Int. Cl. A61K 38/20; C07D 401/14; A61K 39/395; A61P 31/18; A61P 11/06; A61P 19/02; A61P 37/02; A61K 31/445

(52) U.S. Cl. 424/85.2; 546/187; 514/316; 424/145.1

(57) ABSTRACT

This invention relates to piperidine derivatives of formula I wherein R¹, R², R³ and R⁴ are as defined herein useful in the treatment of a variety of disorders, including those in which the modulation of CCR5 receptors is implicated. Disorders that may be treated or prevented by the present derivatives include HIV and genetically related retroviral infections (and the resulting acquired immune deficiency syndrome, AIDS), rheumatoid arthritis, solid organ transplant reject (graft vs. host disease), asthma and COPR.

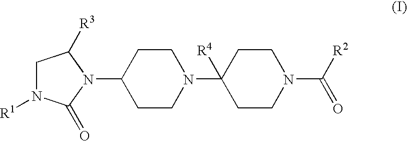

### CROSS REFERENCE TO PRIOR APPLICATIONS

This application claims the benefit of priority to U.S. Ser. No. 60/961,784 filed Jul. 24, 2007 the contents of which are hereby incorporated in their entirety by reference.

### FIELD OF THE INVENTION

This invention relates to piperidine derivatives useful in the treatment of a variety of disorders in which modulation of the CCR5 receptor ligand binding is beneficial. More particularly, to [1,4′]bipiperidinyl-4-yl]-imidazolidin-2-one or 4′-alkyl-[1,4′]bipiperidinyl-4-yl]-imidazolidin-2-one compounds, to compositions containing said compounds and to uses of such derivatives. Disorders that may be treated or prevented by the present compounds include HIV-1 and genetically related retroviral infections (and the resulting acquired immune deficiency syndrome, AIDS), arthritis, asthma, chronic obstructive pulmonary disease (COPD) and rejection of transplanted organs.

### BACKGROUND OF THE INVENTION

Compounds of the present invention modulate the activity of the chemokine CCR5 receptors. The CCR5 receptor is a member of a subset of a large family chemokine receptors characterized structurally by two adjacent cysteine residues. Human chemokines include approximately 50 small proteins of 50-120 amino acids that are structurally homologous. (M. Baggiolini et al., Ann. Rev. Immunol. 1997 15:675-705) The chemokines are pro-inflammatory peptides that are released by a wide variety of cells such as macrophages, monocytes, eosinophils, neutrophiles, fibroblasts, vascular endothelial cells, smooth muscle cells, and mast cells, at inflammatory sites (reviewed in Luster, New Eng. J Med. 1998 338:436-445 and Rollins, Blood 1997 90:909-928). The name “chemokine” is a contraction of “chemotactic cytokines”. The chemokines are a family of leukocyte chemotactic proteins capable of attracting leukocytes to various tissues, which is an essential response to inflammation and infection. Chemokines can be grouped into two subfamilies, based on whether the two amino terminal cysteine residues are immediately adjacent (CC family) or separated by one amino acid (CXC family). The CXC chemokines, such as interleukin-8 (IL-8), neutrophil-activating protein-2 (NAP-2) and melanoma growth stimulatory activity protein (MGSA) are chemotactic primarily for neutrophils and T lymphocytes, whereas the CC chemokines, such as RANTES (CCL5), MIP-1α (CCL3, macrophage inflammatory protein), MIP-1β (CCL4), the monocyte chemotactic proteins (MCP-1, MCP-2, MCP-3, MCP4, and MCP-5) and the eotaxins (−1 and −2) are chemotactic for, among other cell types, macrophages, T lymphocytes, eosinophils, dendritic cells, and basophils. Naturally occurring chemokines which can stimulate the CCR5 receptor include MIP-1α, MIP-1β and RANTES.

Accordingly, drugs which inhibit the binding of chemokines such as MIP-1α, MIP-1β and RANTES to these receptors, e.g., chemokine receptor antagonists, may be useful as pharmaceutical agents which inhibit the action of chemokines such as MIP-1α, MIP-1β and RANTES on the target cells. The identification of compounds that modulate the finction of CCR5 represents an excellent drug design approach to the development of pharmacological agents for the treatment of inflammatory conditions and diseases associated with CCR5 receptor. The pharmacokinetic challenges associated with large molecules, proteins and peptides resulted in the establishment of programs to identify low molecular weight antagonists of CCR5. The efforts to identify chemokine modulators have been reviewed (W. Kazmierski et al. Biorg Med. Chem. 2003 11:2663-76; L. Agrawal and G. Alkhatib, Expert Opin. Ther. Targets 2001 5(3):303-326; Chemokine CCR5 antagonists incorporating 4-aminopiperidine scaffold, Expert Opin. Ther. Patents 2003 13(9):1469-1473; M. A. Cascieri and M. S. Springer, Curr. Opin. Chem. Biol. 2000 4:420-426, and references cited therein).

In U.S. Patent Publication 20050176703 published Aug. 11, 2005 S. D. Gabriel et al. disclosed 1-oxa-3,8-diaza-spiro[4.5]decan-2-one and 1-oxa-3,9-diaza-spiro[5.5]undecan-2-one derivatives which are CCR5 receptor antagonists.

### SUMMARY OF THE INVENTION

The present invention relates to a compound according to formula I and pharmaceutical compositions comprising a compound according to formula I admixed with at least one carrier, diluent or excipient wherein:

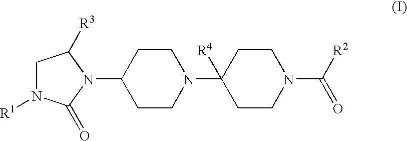

R¹ is: (a) C₃₋₆ cycloalkyl wherein said cycloalkyl is optionally substituted with one to three groups independently selected from the group consisting of hydroxy, C₁₋₃ alkyl, oxo, halogen and C₁₋₆ alkoxy wherein any carbon atom adjacent only to other carbon atoms can be replaced by an oxygen atom;

(b) C₃₋₆ cycloalkyl-C₁₋₃ alkyl wherein said cycloalkyl is optionally substituted with one to three groups independently selected from the group consisting of hydroxy, C₁₋₃ alkyl, oxo, halogen and C₁₋₆ alkoxy wherein any carbon atom adjacent only to other carbon atoms can be replaced by an oxygen atom;

(c) tetrahydropyranyl, tetrahydropyranylmethyl, tetrahydrofuranyl, tetrahydrofuranylmethyl, or [1,4]dioxanyl;

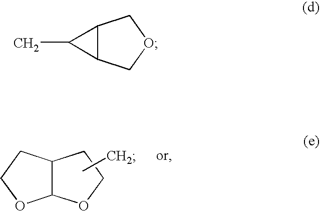

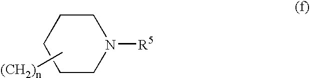

wherein R⁵ is C₁₋₆ acyl, C₁₋₆ alkoxycarbonyl, C₁₋₆ alkyl-SO₂, C₁₋₆ haloalkyl, C₃₋₆ cycloalkyl, carbamoyl, C₁₋₃ alkylcarbamoyl, tetrahydrofuranyl or tetrahydropyranyl and n is 1-3;

R³ is C₁₋₆ alkyl, pyridinyl or phenyl optionally substituted with 1 to 3 halogens

R⁴ is hydrogen or C₁₋₃ alkyl;

R² is selected from the group consisting of (a) to (e) and (f):
(a) 4,6-dimethyl-pyrimidin-5-yl;
(b) 2,4-dimethyl-pyridin-3-yl;
(c) 2,4-dimethyl-1-oxy-pyridin-3-yl
(d) 6-cyano-2,4-dimethyl-pridin-3-yl;
(e) 2,4-dimethyl-6-oxo-1,6-dihydro-pyridin-3-yl;
(f) 4,6-dimethyl-2-trifluoromethyl-pyrimidin-5-yl; or,
(g) 3-methyl-5-trifluoromethyl-isoxazol4-yl; or,

a pharmaceutically acceptable acid addition salt thereof.

The present invention further relates to a method for treating an HIV-1 infection by administering a compound of formula I, either alone or in combination with one or more compounds which inhibit replication of HIV-1. Other mechanistic classes that inhibit HIV-1 include reverse transcriptase inhibitors, protease inhibitors, and viral fusion inhibitors.

The present invention also relates to a method for treating arthritis utilizing a compound of formula I, either alone or in combination with other anti-inflammatory agents useful for alleviation of arthritis.

The present invention additionally relates to a method for treating inflammatory diseases of the lung and airways including asthma and chronic obstructive pulmonary disease (COPD).

The present invention further relates a method for treating transplant rejection utilizing a compound of formula I, either alone or in combination with other anti-rejection drugs or immune system modulators.

The combination therapy utilizing the present compounds can be accomplished with both low-molecular weight compounds and with monoclonal antibodies.

### DETAILED DESCRIPTION OF THE INVENTION

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen.

When any variable (e.g., R¹, R^{4a}, Ar, X¹ or Het) occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or “-----” drawn through a bond each refer to the point of attachment of a functional group or other chemical moiety to the rest of the molecule of which it is a part. Thus, for example:

MeC(═O)OR⁴ wherein

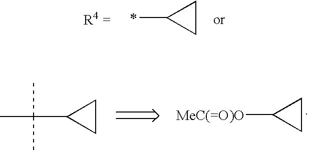

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used herein, the recitation of a numerical range for a variable is intended to convey that the invention may be practiced with the variable equal to any of the values within that range. Thus, for a variable which is inherently discrete, the variable can be equal to any integer value of the numerical range, including the end-points of the range. Similarly, for a variable which is inherently continuous, the variable can be equal to any real value of the numerical range, including the end-points of the range. As an example, a variable which is described as having values between 0 and 2, can be 0, 1 or 2 for variables which are inherently discrete, and can be 0.0, 0.1, 0.01, 0.001, or any other real value for variables which are inherently continuous.

Compounds of formula I exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

In one embodiment of the present invention there is provided a compound according to formula I wherein R¹, R², R³, R⁴, R⁵ and n are as defined herein above.

In another embodiment of the present invention there is provided a compound according to formula I wherein:

R¹ is: (a) C₃₋₆ cycloalkyl wherein said cycloalkyl is optionally substituted with one to three groups independently selected from the group consisting of hydroxy, C₁₋₃ alkyl, oxo, halogen and C₁₋₆ alkoxy wherein any carbon atom adjacent only to other carbon atoms can be replaced by an oxygen atom;

(b) C₃₋₆ cycloalkyl-C₁₋₃ alkyl wherein said cycloalkyl is optionally substituted with one to three groups independently selected from the group consisting of hydroxy, C₁₋₃ alkyl, oxo, halogen and C₁₋₆ alkoxy wherein any carbon atom adjacent only to other carbon atoms can be replaced by an oxygen atom;

(c) tetrahydropyranyl, tetrahydropyranylmethyl, tetrahydrofuranyl, tetrahydrofuranylmethyl, or [1,4]dioxanyl;

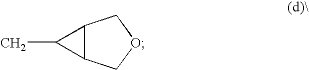

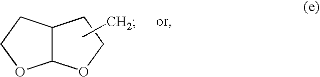

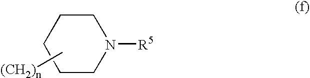

wherein R⁵ is C₁₋₆ acyl, C₁₋₆ alkoxycarbonyl, C₁₋₆alkyl-SO₂, C₁₋₆haloalkyl, C₃₋₆cycloalkyl, carbamoyl, C₁₋₃ alkylcarbamoyl, tetrahydrofuranyl or tetrahydropyranyl and n is 1-3;

R³ is C₁₋₆ alkyl, pyridinyl or phenyl optionally substituted with 1 to 3 halogens

R² is 4,6-dimethyl-pyrimidin-5-yl; 2,4-dimethyl-pyridin-3-yl; 2,4-dimethyl-1-oxy-pyridin-3-yl; 6-cyano-2,4-dimethyl-pridin-3-yl; 2,4-dimethyl-6-oxo-1,6-dihydro-pyridin-3-yl or 4,6-dimethyl-2-trifluoromethyl-pyrimidin-5-yl

In a second embodiment of the present invention there is provided a compound according to formula I wherein R³ is C₃₋₅ alkyl and R⁴ is methyl.

In a third embodiment of the present invention there is provided a compound according to formula I wherein R³ is optionally substituted phenyl or pyridinyl and R⁴ is methyl.

In a fourth embodiment of the present invention there is provided a compound according to formula I wherein R¹ is tetrahydropyranyl or tetrahydropyranylmethyl; R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl; R³ is C₃₋₅ alkyl; and, R⁴ is methyl.

In another embodiment of the present invention there is provided a compound according to formula I wherein R¹ is tetrahydropyran-4-yl or tetrahydropyran-4-ylmethyl; R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl; R³ is C₃₋₅ alkyl; and, R⁴ is methyl.

In still another embodiment of the present invention there is provided a compound according to formula I wherein R¹ is tetrahydrofuran-3-yl or tetrahydrofuran-3-ylmethyl; R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl; R³ is C₃₋₅ alkyl; and, R⁴ is methyl.

In a fifth embodiment of the present invention there is provided a compound according to formula I wherein R¹ is 4-(C₁₋₃ alkoxy)-cyclohexyl-methyl, 4,4-difluorocyclohexyl-methyl, 4-oxo-cyclohexyl-methyl or 4-hydroxycyclohexyl-methyl; R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl; R³ is C₃₋₅ alkyl; and, R⁴ is methyl.

In a sixth embodiment of the present invention there is provided a compound according to formula I wherein R¹ is 4-(C₁₋₃ alkoxy)-cyclohexyl, 4,4-difluorocyclohexyl, 4-oxo-cyclohexyl or 4-hydroxycyclohexyl; R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl; R³ is C₃₋₅ alkyl; and, R⁴ is methyl.

In a seventh embodiment of the present invention there is provided a compound according to formula I wherein R¹ is (i); R³ is C₃₋₅ alkyl; R⁴ is methyl; and, R⁵ is C₁₋₃ acyl, C₁₋₃ alkylsulfonyl, C₁₋₃ haloalkyl or C₁₋₃ alkyl.

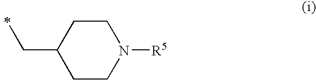

In a another embodiment of the present invention there is provided a compound according to formula I wherein R¹ is (i); R² is 4,6-dimethyl-pyrimidin-5-yl, 2,4-dimethyl-pyridin-3-yl or 6-cyano-2,4-dimethyl-pridin-3-yl; R³ is C₃₋₅ alkyl; R⁴ is methyl; and, R⁵ is acetyl, sulfonylmethyl or 2,2,2-trifluoromethyl.

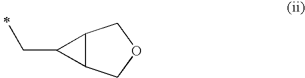

In a eighth embodiment of the present invention there is provided a compound according to formula I wherein R¹ is (ii); R² is 4,6-dimethyl-pyrimidin-5-yl, 2,4-dimethyl-pyridin-3-yl or 6-cyano-2,4-dimethyl-pridin-3-yl; R³ is C₃₋₅ alkyl; and, R⁴ is methyl.

In a ninth embodiment of the present invention there is provided a compound according to formula I wherein R¹ is tetrahydropyranyl, tetrahydropyranylmethyl or tetrahydrofuranylmethyl; R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl; R³ is optionally substituted phenyl or pyridinyl; and, R⁴ is methyl.

In another embodiment of the present invention there is provided a compound according to formula I wherein R¹ is tetrahydropyran-4-yl or tetrahydropyran4-ylmethyl; R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl; R³ is optionally substituted phenyl or pyridinyl; and, R⁴ is methyl.

In still another embodiment of the present invention there is provided a compound according to formula I wherein R¹ is tetrahydrofuran-3-yl or tetrahydrofuran-3-ylmethyl; R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl; R³ is optionally substituted phenyl or pyridinyl and, R⁴ is methyl.

In a tenth embodiment of the present invention there is provided a compound according to formula I wherein R¹ is 4-(C₁₋₃ alkoxy)-cyclohexyl-methyl, 4,4-difluorocyclohexyl-methyl, 4-oxo-cyclohexyl-methyl or 4-hydroxycyclohexyl-methyl; R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl; R³ is optionally substituted phenyl or pyridinyl; and, R⁴ is methyl.

In a eleventh embodiment of the present invention there is provided a compound according to formula I wherein R¹ is 4-(C₁₋₃ alkoxy)-cyclohexyl, 4,4-difluorocyclohexyl, 4-oxo-cyclohexyl or 4-hydroxycyclohexyl; R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl; R³ is optionally substituted phenyl or pyridinyl; and, R⁴ is methyl.

In a twelfth embodiment of the present invention there is provided a compound according to formula I wherein R¹ is (i); R³ is optionally substituted phenyl or pyridinyl; R⁴ is methyl; and, R⁵ is C₁₋₃ acyl, C₁₋₃ alkylsulfonyl, C₁₋₃ haloalkyl or C₁₋₃ alkyl.

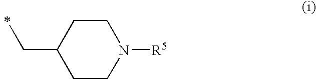

In a thirteenth embodiment of the present invention there is provided a compound according to formula I wherein R¹ is (i); R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl; R³ is optionally substituted phenyl or pyridinyl; R⁴ is methyl; and, R⁵ is C₁₋₃ acyl, C₁₋₃ alkylsulfonyl, C₁₋₃ haloalkyl or C₁₋₃ alkyl.

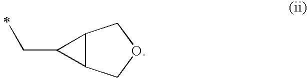

In a fourteenth embodiment of the present invention there is provided a compound according to formula I wherein R¹ is (ii); R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl; ; R³ is optionally substituted phenyl or pyridinyl; and, R⁴ is methyl.

In a fifteenth embodiment of the present invention there is provided a compound according to formula I which compound is I-1 to I-27 in TABLE I or pharmaceutically acceptable salts thereof.

In a sixteenth embodiment of the present invention there is provided a method for treating or preventing an human immunodeficiency virus (HIV-1) infection, or treating AIDS or ARC, in a patient in need thereof which comprises administering to the patient in need thereof a therapeutically effective amount of a compound according to formula I wherein R¹, R², R³, R⁴, R⁵ and n are as defined herein above.

In a seventeenth embodiment of the present invention there is provided a method for treating or preventing an human immunodeficiency virus (HIV-1) infection, or treating AIDS or ARC, in a patient in need thereof which comprises co-administering a therapeutically effective amount of one or more anti-HIV-1 drugs selected from the group consisting of HIV-1 nucleoside reverse transcriptase inhibitors, non-nucleoside reverse transcriptase inhibitors, HIV-1 protease inhibitors, an integrase inhibitor and a HIV-1 viral fusion inhibitors along with a therapeutically effective amount of a compound according to formula I wherein R¹, R², R³, R⁴, R⁵ and n are as defined herein above.

In an eighteenth embodiment of the present invention there is provided a method for treating rheumatoid arthritis comprising administering a therapeutically effective amount of a compound according to formula I wherein R¹, R², R³, R⁴, R⁵ and n are as defined herein above.

In an nineteenth embodiment of the present invention there is provided a method for treating rheumatoid arthritis comprising co-administering one or more anti-inflammatory or analgesic compounds and a therapeutically effective amount of a compound according to formula I wherein R¹, R², R³, R⁴, R⁵ and n are as defined herein above.

In an twentieth embodiment of the present invention there is provided a method for treating asthma or congestive obstructive pulmonary disease (COPD) comprising administering a therapeutically effective amount of a compound according to formula I wherein R¹, R², R³, R⁴, R⁵ and n are as defined herein above.

In an twenty-first embodiment of the present invention there is provided a method for treating solid organ transplant rejection comprising administering a therapeutically effective amount of a compound according to formula I wherein R¹, R², R³, R⁴, R⁵ and n are as defined herein above.

In an twenty-second embodiment of the present invention there is provided a method for treating solid organ transplant rejection comprising co-administering a therapeutically effective amount of one or more anti-rejection drugs or immunomodulators along with a therapeutically effective amount of a compound according to formula I wherein R¹, R², R³, R⁴, R⁵ and n are as defined herein above.

In a twenty-fourth embodiment of the present invention there is provided a pharmaceutical composition containing a compound according to formula I wherein R¹, R², R³, R⁴, R⁵ and n are as defined herein above and at least one carrier, diluent or excipient.

### Methods for Treating HIV-1 Infections

HIV-1 infects cells of the monocyte-macrophage lineage and helper T-cell lymphocytes by exploiting a high affinity interaction of the viral enveloped glycoprotein (Env) with the CD4 antigen. The CD4 antigen was found to be a necessary, but not sufficient requirement for cell entry and at least one other surface protein was required to infect the cells (E. A. Berger et al., Ann. Rev. Immunol. 1999 17:657-700). Two chemokine receptors, either the CCR5 or the CXCR4 receptor, were subsequently found to be co-receptors along with CD4 which are required for infection of cells by the human immunodeficiency virus (HIV). The central role of CCR5 in the pathogenesis of HIV was inferred by epidemiological identification of powerful disease modifying effects of the naturally occurring null allele CCR5 Δ32. The Δ32 mutation has a 32-base pair deletion in the CCR5 gene resulting in a truncated protein designated Δ32. Relative to the general population, Δ32/Δ32 homozygotes are significantly common in exposed/uninfected individuals suggesting the role of CCR5 in HIV cell entry (R. Liu et al., Cell 1996 86(3):367-377; M. Samson et al., Nature 1996 382(6593):722-725). The CD-4 binding site on the gp120 of HIV appears to interact with the CD4 molecule on the cell surface resulting in a conformational change that allows it to bind to either the CCR5 and/or CXCR-4 cell-surface receptor. This brings the viral envelope closer to the cell surface and allows interaction between gp41 on the viral envelope and a fusion domain on the cell surface, fusion with the cell membrane, and entry of the viral core into the cell. Accordingly, an agent which could block chemokine receptors in humans who possess normal chemokine receptors should prevent infection in healthy individuals and slow or halt viral progression in infected patients.

RANTES and an analog chemically modified on the N-terminus, aminooxypentane RANTES, were found to block HIV entry into the cells. (G. Simmons et al., Science 1997 276:276-279). Other compounds have been demonstrated to inhibit the replication of HIV, including soluble CD4 protein and synthetic derivatives (Smith, et al., Science 1987 238:1704-1707), dextran sulfate, the dyes Direct Yellow 50, Evans Blue, and certain azo dyes (U.S. Pat. No. 5,468,469). Some of these antiviral agents have been shown to act by blocking the binding of gp120, the coat protein of HIV, to its target, the CD4 glycoprotein of the cell.

A-M. Vandamme et al. (Antiviral Chem. & Chemother., 1998 9:187-203) disclose current HAART clinical treatments of HIV-1 infections in man including at least triple drug combinations. Highly active anti-retroviral therapy (HAART) has traditionally consisted of combination therapy with nucleoside reverse transcriptase inhibitors (NRTI), non-nucleoside reverse transcriptase inhibitors (NNRTI) and protease inhibitors (PI). These compounds inhibit biochemical processes required for viral replication. While HAART has dramatically altered the prognosis for HIV infected persons, there remain many drawbacks to the current therapy including highly complex dosing regimes and side effects which can be very severe (A. Carr and D. A. Cooper, Lancet 2000 356(9239):1423-1430). Moreover, these multidrug therapies do not eliminate HIV-1 and long-term treatment usually results in multidrug resistance, thus limiting their utility in long term therapy. Development of new therapeutics which can be used in combination with NRTIs, NNRTIs, PIs and viral fusion inhibitors to provide better HIV-1 treatment remains a priority.

Typical suitable NRTIs fro HAART therapy include zidovudine (AZT; RETROVIR®); didanosine (ddl; VIDEX®); zalcitabine (ddC; HIVID®); stavudine (d4T; ZERIT®); lamivudine (3TC; EPIVIR®); abacavir (ZIAGEN®); adefovir dipivoxil [bis(POM)-PMEA; PREVON®]; lobucavir (BMS-180194), a nucleoside reverse transcriptase inhibitor disclosed in EP-0358154 and EP-0736533; BCH-10652, a reverse transcriptase inhibitor (in the form of a racemic mixture of BCH-10618 and BCH-10619) under development by Biochem Pharma; emitricitabine [(−)-FTC] in development by Triangle Pharmaceuticals; β-L-FD4 (also called β-L-D4C and named β-L-2′,3′-dicleoxy-5-fluoro-cytidene) licensed Vion Pharmaceuticals; DAPD, the purine nucleoside, (−)-β-D-2,6-diamino-purine dioxolane disclosed in EP-0656778 and licensed to Triangle Pharmaceuticals; and lodenosine (FddA), 9-(2,3-dideoxy-2-fluoro-β-D-threo-pentofuranosyl)adenine, an acid stable purine-based reverse transcriptase inhibitor under development by U.S. Bioscience Inc.

Typical suitable NNRTIs include nevirapine (BI-RG-587; VIRAMUNE®); delaviradine (BHAP, U-90152; RESCRIPTOR®); efavirenz (DMP-266; SUSTIVA®); PNU-142721, a furopyridine-thio-pyrimidine under development by Pfizer; AG-1549 (formerly Shionogi # S-1153); 5-(3,5-dichlorophenyl)-thio-4-isopropyl-1-(4-pyridyl)methyl-1H-imidazol-2-ylmethyl carbonate disclosed in WO 96/10019; MKC-442 (1-(ethoxy-methyl)-5-(1-methylethyl)-6-(phenylmethyl)-(2,4(1H, 3H)-pyrimidinedione); and (+)-calanolide A (NSC-675451) and B, coumarin derivatives disclosed in U.S. Pat. No. 5,489,697.

Typical suitable PIs include saquinavir (Ro 31-8959; INVIRASE®; FORTOVASE®); ritonavir (ABT-538; NORVIR®); indinavir (MK-639; CRIXIVAN®); nelfnavir (AG-1343; VIRACEPT®); amprenavir (141W94; AGENERASE®); lasinavir (BMS-234475); DMP-450, a cyclic urea under development by Triangle Pharmaceuticals; BMS-2322623, an azapeptide under development by Bristol-Myers Squibb as a 2nd-generation HIV-1 PI; ABT-378 under development by Abbott; and AG-1549 an imidazole carbamate under development by Agouron Pharmaceuticals, Inc.

Other antiviral agents include hydroxyurea, ribavirin (1-β-D-ribofuranosyl-1H-1,2,4-triazole-3-carboxamide), IL-2, IL-12, pentafuside. Hydroxyurea (Droxia), a ribonucleoside triphosphate reductase inhibitor shown to have a synergistic effect on the activity of didanosine and has been studied with stavudine. IL-2 (aldesleukin; PROLEUKIN®) is disclosed in Ajinomoto EP-0142268, Takeda EP-0176299, and Chiron U.S. Pat. Nos. RE 33,653, 4,530,787, 4,569,790, 4,604,377, 4,748,234, 4,752,585, and 4,949,314. Pentafuside (FUZEON®) a 36-amino acid synthetic peptide that inhibits fusion of HIV-1 to target membranes. Pentafuside (3-100 mg/day) is given as a continuous sc infusion or injection together with efavirenz and 2 PI's to HIV-1 positive patients refractory to a triple combination therapy; use of 100 mg/day is preferred.

In addition to the potential for CCR5 modulators in the management of HIV infections, the CCR5 receptor is an important regulator of immune finction and compounds of the present invention may prove valuable in the treatment of disorders of the immune system. Treatment of solid organ transplant rejection, graft v. host disease, rheumatoid arthritis, inflammatory bowel disease, atopic dermatitis, psoriasis, asthma, allergies or multiple sclerosis by administering to a patient in need of such treatment an effective amount of a CCR5 antagonist compound of the present invention is also possible.

### Methods for Treating Rheumatoid Arthritis

Modulators of the CCR5 receptor may be useful in the treatment of various inflammatory conditions. Rheumatoid arthritis is characterized by infiltration of memory T lymphocytes and monocytes into inflamed joints. As leukocyte chemotactic factors, chemokines play an indispensable role in the attraction of macrophages to various tissues of the body, a process which is essential for both inflammation and the body's response to infection. Because chemokines and their receptors regulate trafficking and activation of leukocytes which contribute to the pathophysiology of inflammatory and infectious diseases, agents which modulate CCR5 activity, preferably antagonizing interactions of chemokines and their receptors, are useful in the therapeutic treatment of such inflammatory diseases.

Elevated levels of CC chemokines, especially CCL2, CCL3 and CCL5, have been found in the joints of patients with rheumatoid arthritis and have been correlated with the recruitment on monocytes and T cells into synovial tissues (I. F. Charo and R. M. Ransohoff, New Eng J. Med. 2006 354:610-621). T-cells recovered from synovial fluid of rheumatoid arthritis have been shown to express CCR5 and CXCR3. P. Gao et al., J. Leukocyte Biol. 2003 73:273-280) Met-RANTES is an amino-terminal modified RANTES derivative which blocks RANTES binding to the CCR1 and CCR5receptors with nanomolar potency. (A. E. Proudfoot et al., J. Biol. Chem. 1996 271:2599-2603). The severity of arthritis in rats adjuvant-induced arthritis was reduced by the administration of Met-RANTES. In addition, the levels of pro-inflammatory cytokines TNF-α and IL-1β were reduced. (S. Shahrara et al. Arthr. & Rheum. 2005 52:1907-1919) Met-RANTES has been shown to ameliorate the development of inflammation in an art recognized rodent model of inflammation, the collagen induced arthritis. (C. Plater-Zyberk et al. Immunol. Lett. 1997 57:117-120)

TAK-779 has also been shown to reduce both the incidence and severity of arthritis in the collagen-induced arthritis model. The antagonist inhibited the infiltration of inflammatory CCR5⁺ T-cells into the joint. (Y.-F. Yang et al., Eur. J. Immunol. 2002 32:2124-2132). Another CCR5 antagonist, SCH-X, was shown to reduce the incidence and severity of collagen-induced arthritis in rhesus monkeys. (M. P. M. Vierboom et al., Arthr. & Rheum. 2005 52(20):627-636).

In some anti-inflammatory conditions compounds of the present invention may be administered in combination with other anti-inflammatory drugs which may have a alternative mode of action. Compounds which may be combined with CCR5 antagonists include, but are not limited to:

(a) lipoxygenase antagonist or biosynthesis inhibitor such as an inhibitor of 5-lipoxygenase, (b) leukotriene antagonists (e.g., zafirlukast, montelukast, pranlukast, iralukast, pobilukast, SKB-106,203), (c) leukotriene biosynthesis inhibitors (e.g., zileuton, BAY-1005); (d) a non-steroidal antiinflammatory agent or cyclooxygenase (COX1 and/or COX2) inhibitor such as such as propionic acid derivatives (e.g., alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (e.g., indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenarnic acid derivatives (flufenarnic acid, meclofenamic acid, mefenamic acid, niflumic acid and tolfenarnic acid), biphenylearboxylic acid derivatives (diflunisal and flufenisal), oxicarns (isoxicarn, piroxicam, sudoxicam and tenoxican), salicylates (acetyl salicylic acid, sulfasalazine), pyrazolones (apazone, bezpiperylon, feprazone, mofebutazone, oxyphenbutazone, phenylbutazone) and celecoxib; (e) a TNF inhibitor such as infliximab (REMICADE®), etanercept (ENBREL®), or adalimumab (HUMIRA®); (f) anti-inflammatory steroids such as beclomethasone, methylprednisolone, betamethasone, prednisone, dexamethasone, and hydrocortisone; (g) immunomodulators such as cyclosporine, leflunomide (ARAVA®), azathioprine (AZASAN®), penicillamine and levamisole; (h) folate antagonists such as methotrexate; (i) gold compounds such as aurothioglucose, gold sodium thiomalate or auranofin.

### Methods for Treating Transplant Rejection

Rejection following solid organ transplantation also is characterized by infiltration of T-cells and macrophages expressing the CCR5 receptor into the interstitial area. (J. Pattison et al., Lancet 1994 343:209-211) Renal transplant patients homozygous for the CCR5Δ32 deletion a significant survival advantage of patients heterozygous for the CCR5Δ32 deletion or homozygous wild type patients. (M. Fischerder et al., Lancet 2001 357:1758-1761) CCR5^{-/-} knock-out mice showed significant prolong graft survival in after transplantation of heart and islet tissue. (W. Gao et al., Transplantation 2001 72:1199-1205; R. Abdi et al., Diabetes 2002 51:2489-2495. Blocking the CCR5 receptor activation has been found to significantly extend cardiac allograph survival. (W. W. Hancock et al., Curr. Opin. Immunol. 2003 15:479-486).

In treatment of transplant rejection or graft vs. host diseases CCR5 antagonists of the present invention may be administered in combination with other immunosuppressive agents including, but are not limited to, cyclosporine (SANDIMMUNE®), tacrolimus (PROGRAF®, FK-506), sirolimus (RAPAMUNE®, rapamycin), mycophenolate mofetil (CELLCEPT®), methotrexate, anti-IL-2 receptor (anti-CD25) antibodies such as daclizumab (ZENAPAX®) or basiliximab (SIMULECT®), anti-CD3 antibodies visilizumab (NLVION®) or muromonab (OKT3, ORTHOCLONE®).

### Methods for Treating Asthma and COPD

Antagonism of the CCR5 receptor has been suggested as a target to inhibit of progression of asthma and COPD by antagonism of Thl activation: B. Ma et al., J. Immunol. 2006 176(8):4968-4978, B. Ma et al., J. Clin. Investig. 2005 115(12):3460-3472 and J. K. L. Walker et al., Am. J. Respir. Cell Mo. Biol. 2006 34:711-718.

The term “alkyl” as used herein denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁-₁₀ alkyl” as used herein refers to an alkyl composed of 1 to 10 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

The term “alkylene” as used herein denotes a divalent saturated linear hydrocarbon radical of 1 to 8 carbon atoms or a branched saturated divalent hydrocarbon radical of 3 to 8 carbon atoms, unless otherwise indicated. Examples of alkylene radicals include, but are not limited to, methylene, ethylene, propylene, 2-methyl-propylene, butylene and 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defmed. “C₁-₁₀ alkoxy” as used herein refers to an-O-alkyl wherein alkyl is C₁₋₁₀. The term “oxo” as use herein refers to a carbonyl (═O) group. Thus cyclohexane with an oxo substituent is cyclohexanone.

The term “cycloalkyl” as used herein denotes a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term “cycloalkyl alkyl” as used herein refers to the radical R′R″—, wherein R¹ is a cycloaLkyl radical as defmed herein, and R″ is an alkylene radical as defmed herein with the understanding that the attachment point of the cycloalkylalkyl moiety will be on the alkylene radical. Examples of cycloalkylalkyl radicals include, but are not limited to, cyclopropylmethyl, cyclohexylmethyl, cyclopentylethyl. C₃₋₇ cycloalkyl-C₁₋₃ alkyl refers to the radical R′R″ where R¹ is C₃₋₇ cyclolalkyl and R″ is C₁₋₃ alkylene as defined herein.

The term “halogen” or “halo” as used herein means fluorine, chlorine, bromine, or iodine.

The term “haloalkyl” as used herein denotes a unbranched or branched chain alkyl group as defmed above wherein 1, 2, 3 or more hydrogen atoms are substituted by a halogen. “C₁₋₃ haloalkyl” as used herein refers to an haloalkyl composed of 1 to 3 carbons and 1-8 halogen substituents. Examples are 1-fluoromethyl, 1-chloromethyl, 1-bromomethyl, 1-iodomethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, 2,2-dichloroethyl, 3-bromopropyl, 2,2,2-trifluoroethyl or difluoromethyl.

The term “C₁₋₆ fluoroalkyl” as used herein denotes a unbranched or branched chain alkyl group as defined above wherein 1, 2, 3 or more hydrogen atoms are substituted by a fluorine.

The terms “tetrahydrofuranyl” and “tetrahydropyranyl” refer to a five and six-membered non-fused heterocyclic ring respectively, each containing one oxygen atom. The term “pyridine” refers to a six-membered heteroaromatic ring with one nitrogen atom. The term “pyrimidine”, refers to a six-membered nonftised heteroaromatic ring with two nitrogen atoms disposed in a 1,3, relationship. The term 4′-alkyl-[1,4′]bipiperidinyl-4-yl]-imidazolidin-2-one and [1,4′]bipiperidinyl-4-yl]-imidazolindin-2-one refers to a compound of formula (iii) where R⁴ is C₁₋₃ alkyl or hydrogen respectively.

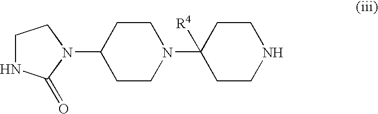

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIALD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppf), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-Azabenzotriazole-1-yl)-N,N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TM S), p-toluenesulfonic acid monohydrate (TsOH or pTsOH), 4-Me-C₆H₄SO₂— or tosyl (Ts), N-urethane-N-carboxyanhydride (UNCA),. Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### Compounds and Invention

Examples of representative compounds encompassed by the present invention and within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE I

| Cpd. | 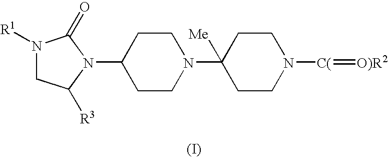 | | | | CCR5Binding¹IC₅₀ |
|---|---|---|---|---|---|
| No/ | R¹ | R² | R³ | ms | (μM) |
| I-1 | 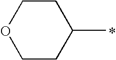 | 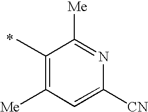 | n-Bu | 565 | 0.0085 |
| I-2 | 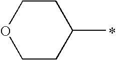 | 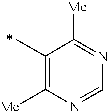 | n-Bu | 541 | 0.9694 |
| I-3 | 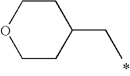 | 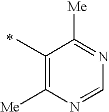 | n-Bu | 555 | 0.1096 |
| I-4 | 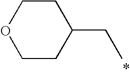 | 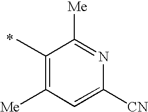 | n-Bu | 579 | 0.0044 |
| I-5 | 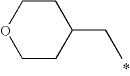 | 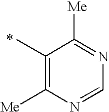 | (R)-Ph | 575 | 0.0237 |
| I-6 | 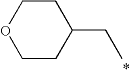 | 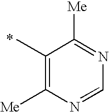 | (R,S)-Ph | 575 | 0.0275 |
| I-7 | 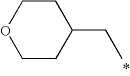 | 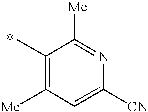 | (R)-Ph | 599 | 0.0112 |
| I-8 | 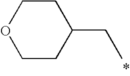 | 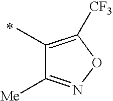 | (R)-Ph | 618 | 0.0051 |
| I-9 | 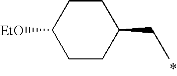 | 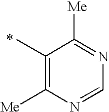 | (R)-Ph | 617 | 0.0126 |
| I-10 | 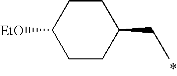 | 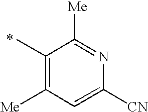 | (R)-Ph | 641 | 0.0106 |
| I-11 | 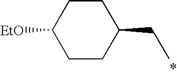 | 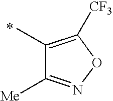 | (R)-Ph | 660 | 0.0068 |
| I-12 | 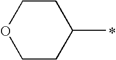 | 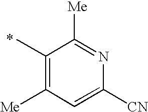 | (R)-Ph | 585 | 0.09 |
| I-13 | 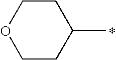 | 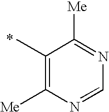 | (R)-Ph | 561 | 0.09 |
| I-14 | 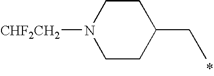 | 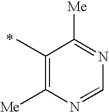 | (R)-Ph | 638 | 0.0264 |
| I-15 | 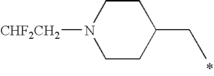 | 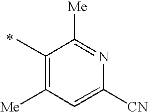 | (R)-Ph | 662 | 0.0183 |
| I-16 | 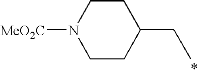 | 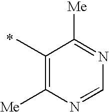 | (R)-Ph | 632 | 0.0258 |
| I-17 | 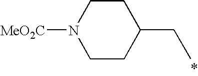 | 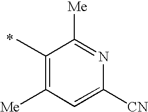 | (R)-Ph | 656 | 0.0357 |
| I-18 | 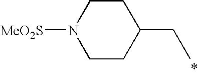 | 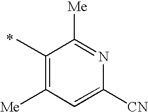 | (R)-Ph | 676 | 0.0366 |
| I-19 | 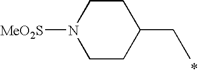 | 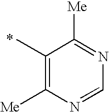 | (R)-Ph | 652 | 0.1167 |
| I-20 | 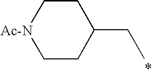 | 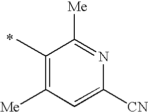 | (R)-Ph | 640 | 0.0425 |
| I-21 | 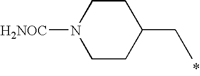 | 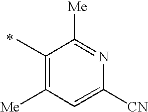 | (R)-Ph | 641 | 0.044 |
| I-22 | 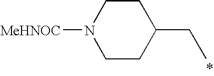 | 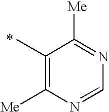 | (R)-Ph | 630 | 0.0318 |
| I-23 | 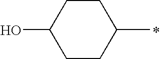 | 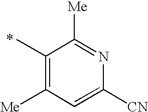 | (R)-Ph | 599 | 0.0206 |
| I-24 | 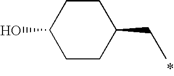 | 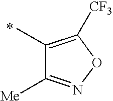 | (R)-Ph | 632 | 0.0071 |
| I-25 | 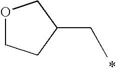 | 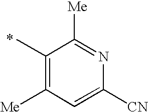 | (R)-Ph | 585 | 0.0544 |

| | | | |
|---|---|---|---|
| I-26 | 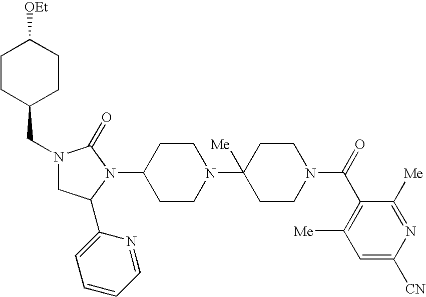 | 642 | 0.0247 |
| I-27 | 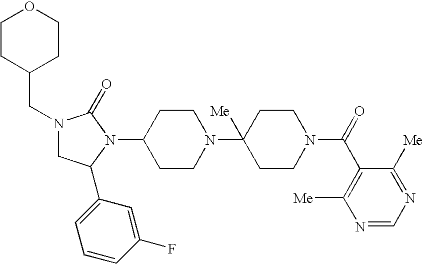 | 593 | 0.0894 |

1. CCR5 binding assay is example 6

Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, Volumes 1-21; R. C. LaRock, Comprehensive Organic Transformations, 2^{nd} edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40. The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

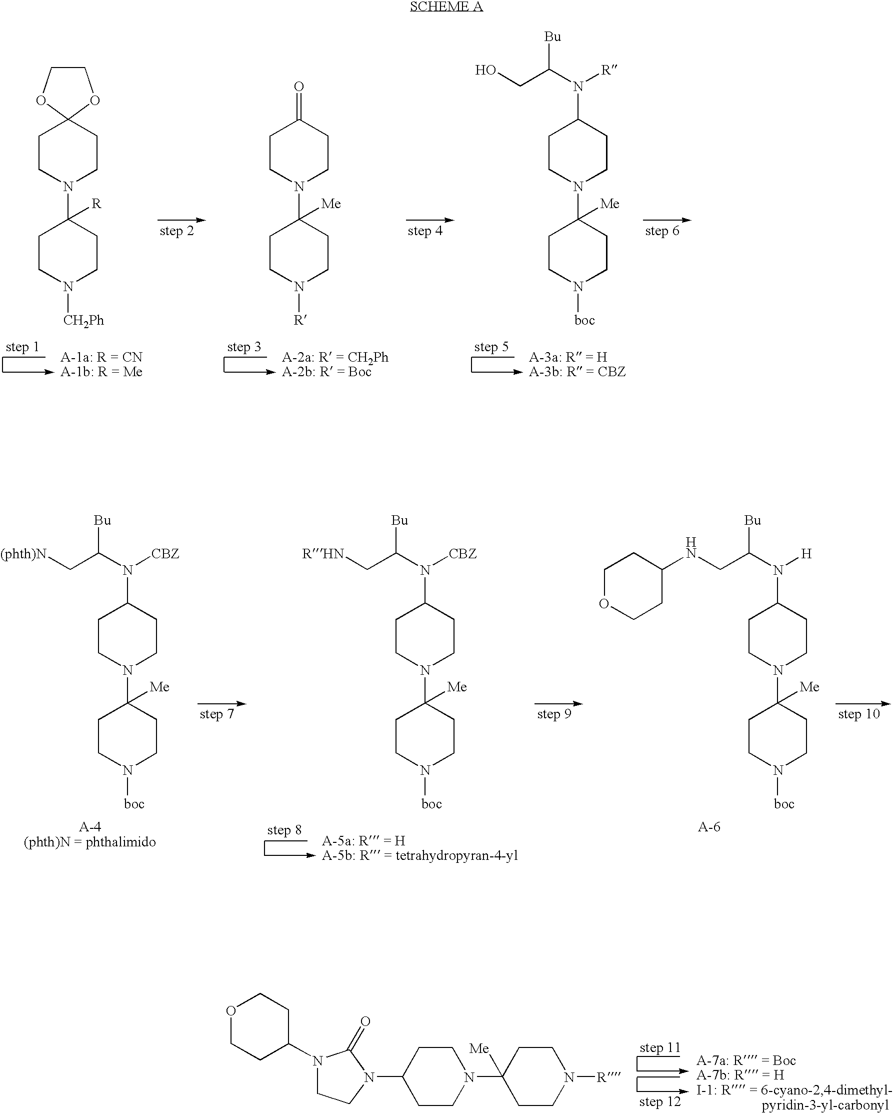

Some compounds in following schemes are depicted with generalizedsubstituents; however, one skilled in the art will immediately appreciate that the nature and number of the R groups can varied to afford the various compounds contemplated in this invention. The general formulae in the schemes are intended to be illustrative and are not intended to imply a limitation to the scope of the invention which is defined by the appended claims. Moreover, the reaction conditions are exemplary and alternative conditions are well known. The reaction sequences in the following examples are not meant to limit the scope of the invention as set forth in the claims.

The imidazolidin-2-one derivatives of formula I can be elaborated from A-2b (CASRN 438208-23-2). Contacting A-2b with a vicinal amino alcohol substituted on the nitrogen-bearing carbon with an alkyl, phenyl or heteroaryl moiety under conditions suitable for reductive amination affords an amino alcohol of formula A-3a which can be cyclized to produce the imidazolidinone ring. Reductive amination is typically carried out by combining an amine and carbonyl compound in the presence of a complex metal hydride such as NaBH₄, LiBH₄, NaBH₃CN, Zn(BH₄)₂, sodium triacetoxyborohydride or borane/pyridine conveniently at a pH of 1-7 optionally in the presence of a dehydrating agent such as molecular sieve or Ti(IV)(O-i-Pr)₄ to facilitate formation of the intermediate imine at ambient temperature. Alternatively the imine can be formed under a hydrogen atmosphere in the presence of a hydrogenation catalyst, e.g. Pd/C, at a hydrogen pressure of 1 to 5 bar, preferably at temperatures between 20° C. and the boiling temperature of the solvent used. It may also be advantageous during the reaction if reactive groups are protected during the reaction by conventional protecting groups which are cleaved again by conventional methods after the reaction. Reductive amination procedures have been reviewed: R. M. Hutchings and M. K. Hutchings Reduction of C═N to CHNH by Metal Hydrides in Comprehensive Organic Synthesis col. 8, I. Fleming (Ed) Pergamon, Oxford 1991 pp. 47-54.

After the nitrogen is protected the second nitrogen atom of the imidazolidinone ring is incorporated by displacement of the alcohol. This can conventionally be done by converting the alcohol to a leaving group such as a halogen, a mesylate (methanesulfonyloxy) or tosylate and contacting that compound with ammonia or an ammonia equivalent such as a phthalimide salt or an azide salt (the azide being subsequently reduced to the amine). In the present example a phthalimide is used to displace the hydroxy group under Mitsunobu conditions (D. L. Hughes, The Mitsunobu Reaction, in Organic Reactions, Volume 42, 1992, John Wiley & Sons, New York; pp. 335-656) which comprise activating alcohols with a mixture of a phosphine such as a trialkylphosphine like (n-Bu)₃P or a triarylphosphine like Ph₃P and a diazo-compound like diethyl-azodicarboxylate (DEAD), diisopropyl-azodicarboxylate (DIAD) or di-tert-butyl-azodicarboxylate in a inert solvent commonly used for such transformations such as THF, toluene or DCM. There is no particular restriction on the nature of the solvent to be employed, provided that it has no adverse effect on the reaction or the reagents involved and that it can dissolve the reagents, at least to some extent. The reaction can take place over a wide range of temperatures ranging from ambient temperatures to the reflux temperature of the solvent employed. The phthalimide was removed by treatment with hydrazine under standard conditions (Green and Wuts, supra).

Phenyl substitution on the imidazolidin-2-one ring was accomplished by a variation of the route depicted in SCHEME A and is depicted in SCHEME B wherein an amino azide is elaborated prior to the reductive amination with A-2b. The requisite amine, 2-azido-1-phenyl-ethylamine, was prepared from B-1a which is readily available from phenylglycine. One skilled in the art will appreciate that chiral amino can be readily prepared (R. M. Williams, In Synthesis of Optically Active α-Amino Acids; Baldwin, J. E., Ed.; Organic Chemistry Series; Pergamon Press: Oxford, 1989; R. M. Williams and J. A. Hendrix, Chem. Rev. 1992 92:889; R. O. Duthaler, R. O. Tetrahedron 1994 50:1539; D. Seebach et al., Angew. Chem., Int. Ed. Engl. 1996 35:27081; V. Cativiela et al. Tetrahedron: Asymmetry 1998 9:3517) and compounds encompassed in the present invention where the imidazolinone is substituted by substituted aryl or other groups are accessible by analogous procedures from the appropriate amino acid.

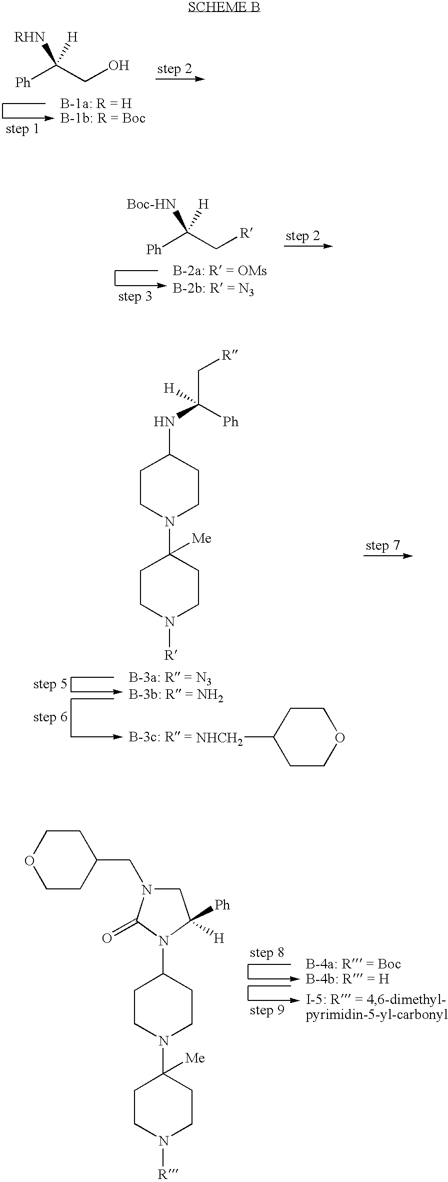

Reduction of the amino acid to the corresponding amino alcohol, selective N-protection with Boc, activation of the alcohol for displace by mesylation and displacement of the methanesulfonic acid with sodium azide (steps 1-3) in SCHEME B is accomplished by well known procedures. The Boc group is removed with TFA/DCM and the resulting amine is subjected to reductive alkylation with A-2b to afford B-3a. Reduction of the azide was conveniently carried out by catalytic reduction to afford B-3b which was then converted to B-3c be reductive alkylation with pyran-4-carboxaldehyde. Closure of the imidazolidinone ring and incorporation of the pyrimidine-carboxamide was accomplished as depicted in SCHEME A.

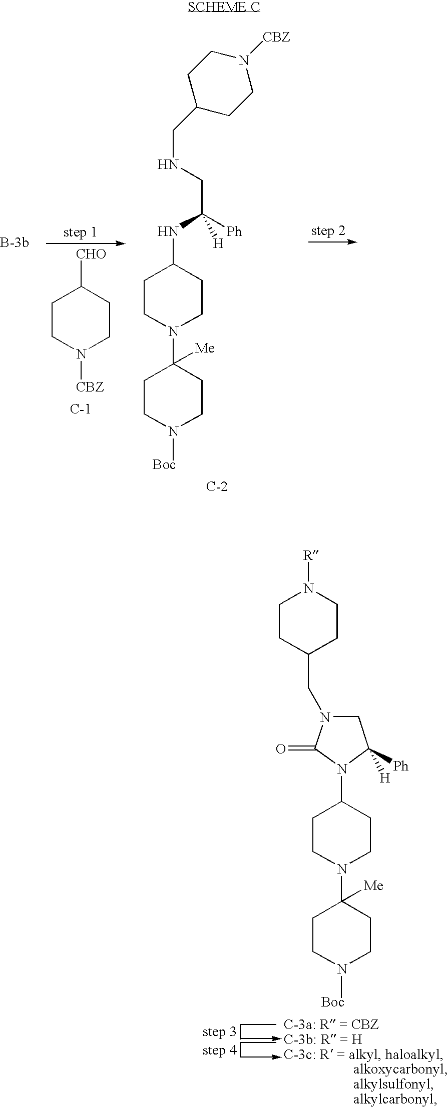

Compounds encompassed by the present invention containing an N-substituted piperidin-4-ylmethyl moiety were accessible through the common intermediate C-3b. Reductive alkylation of C-1 (CASRN 138163-08-3) and B-3b affords the diamine C-2 which is cyclized to the imidazolidinone with phosgene or an equivalent such as bis-(trichloromethyl)carbonate, trichloromethyl-chloro-formate, carbonyldiimidazole, polycarbonate and the like to afford C-3a. Selective removal of the CBZ protecting group was carried out using catalytic hydrogenation which permits alkylation, acylation or sulfonylation of the amine to afford C-3c. removal of the Boc protecting group and acylation of the other piperidine nitrogen is the carried out as previously described.

One skilled in the art will appreciate the sequence of reactions as well as the particular conditions employed are exemplary and the sequence can be altered or the reaction conditions modified without departing from the spirit of the invention.

### Biological Assays

The capacity for novel compounds of the present invention to bind to the CCR5 receptor and thereby antagonize CCR5 function can be evaluated with assay systems known in the art (example 6). The capacity of compounds of the present invention to inhibit infection of CD4⁺/CCR5⁺ expressing cells can be determined using a cell-cell fusion assay as described in example 7 or an antiviral assay as described in example 8.

Functional assays directly measure the ability of a compound to produce a biologically relevant response or inhibit a response produced by a natural ligand (i.e., characterizes the agonist vs. antagonist properties of the test compounds). In a calcium flux assay, cells expressing the CCR5 are loaded with calcium sensitive dyes prior to addition of compound or the natural CCR5 ligand. Compounds with agonist properties will induce a calcium flux signal in the cell, while the compounds of this invention are identified as compounds which do not induce signaling by themselves but are capable of blocking signaling by the natural ligand RANTES.

The chemotaxis assay is a functional assay which measures the ability of a non-adherent cell line expressing human CCR5 receptor to migrate across a membrane in response to either test compounds or natural attractant ligand(s) (i.e., RANTES, MIP-1β). Generally, chemotaxis assays monitor the directional movement or migration of a suitable cell (such as a leukocyte (e.g., lymphocyte, eosinophil, basophil)) into or through a barrier (e.g., endothelium, a permeable filter membrane), toward, from a first surface of the barrier toward an opposite second surface containing attractant ligands. Membranes or filters provide convenient barriers to monitor the directional movement or migration of a suitable cell into or through a filter, toward increased levels of an attractant. In some assays, the membrane is coated with a substance to facilitate adhesion, such as ICAM-1, fibronectin or collagen. Such assays provide an in vitro approximation of leukocyte “homing”. Compounds that are antagonists not only fail to induce chemotaxis, but are also capable of inhibiting cell migration in response to known CCR5 ligands.

A suitable membrane, having a suitable pore size for monitoring specific migration in response to compound, including, for example, nitrocellulose, polycarbonate, is selected. For example, pore sizes of about 3-8 microns, and preferably about 5-8 microns can be used. The pore size can be uniform on a filter or within a range of suitable pore sizes.

To assess migration and inhibition of migration, the distance of migration into the filter, the number of cells crossing the filter that remain adherent to the second surface of the filter, and/or the number of cells that accumulate in the second chamber can be determined using standard techniques (e.g., microscopy). In one embodiment, the cells are labeled with a detectable label (e.g., radioisotope, fluorescent label, antigen or epitope label), and migration can be assessed in the presence and absence of the antibody by determining the presence of the label adherent to the membrane and/or present in the second chamber using an appropriate method (e.g., by detecting radioactivity, fluorescence, immunoassay).

In a more physiologically relevant variation of a chemotaxis assay, particularly for T cells, monocytes or cells expressing a mammalian CCR5, transendothelial migration is monitored. Such assays mimic leukocytes migration from blood vessels toward chemoattractants present in the tissues at sites of inflammation by crossing the endothelial cell layer lining the vessel wall.

Endolthelial cells can be cultured and form a confluent layer on a microporous filter or membrane, optionally coated with a substance such as collagen, fibronectin, or other extracellular matrix proteins, to facilitate the attachment of endothelial cells. A variety of mammalian endothelial cells can are available for monolayer formation, including for example, vein, artery or microvascular endothelium. Generally, the assay is performed by detecting the directional migration of cells into or through a membrane or filter.

In a composition comprising cells capable of migration and expressing a mammalian CCR5 receptor can be placed in the first chamber. A composition comprising one or more natural attractant ligands capable of inducing chemotaxis of the cells in the first chamber is placed in the second chamber. Preferably shortly before the cells are placed in the first chamber, or simultaneously with the cells, a composition comprising the compound to be tested is placed, preferably, in the first chamber. Compounds which can bind receptor and inhibit the induction of chemotaxis by natural attractant ligands, of the cells expressing a mammalian CCR5 are inhibitors of receptor function. A reduction in the extent of migration induced by the ligand or promoter in the presence of the antibody is indicative of inhibitory activity. An example of a chemotaxis assay is described in example 8.

Phosphoflow cytometry is a method used to detect phosphorylated proteins in single cells using a phospho specific antibody detected by a flow cytometer. (Peter O. Krutzik et al., Clinical Immunology 2004 110(3):206; P. O. Krutzik et al., Methods Mol Biol.2004 263:67) This is different from traditional flow cytometry, which is used primarily to detect and quantify the presence of surface molecules on cells or the presence of intracellular molecules such as cytokines, relative to control populations of other cells. Phosphoflow on the other hand detects activation induced changes of signaling molecules inside the cell relative to control unstimulated populations of identical cells which may or may not be in the same sample. The phosphoflow cytometry assay is described in example 9.

### Dosage and Administration

The compounds of the present invention may be formulated in a wide variety of oral administration dosage forms and carriers. Oral administration can be in the form of tablets, coated tablets, dragees, hard and soft gelatine capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It should be understood that all references to pharmaceutically acceptable salts include solvent addition forms (solvates) or crystal forms (polymorphs) as defmed herein, of the same acid addition salt.

“Pharmaceutically acceptable” means that the moiety is useful in preparing a pharmaceutical composition that is generally safe, non-toxic, and neither biologically nor otherwise undesirable.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the fmely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to a skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyactic acid.

Suitable formulations along with pharmaceutical carriers, diluents and expcipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

In embodiments of the invention, the active compound or a salt can be administered in combination with one or more antiviral agents, such as a nucleoside reverse transcriptase inhibitor, another nonnucleoside reverse transcriptase inhibitor, a HIV protease inhibitor or a viral entry inhibitor. When the active compound or its derivative or salt are administered in combination with another antiviral agent the activity may be increased over the parent compound. When the treatment is combination therapy, such administration may be concurrent or sequential with respect to that of the nucleoside derivatives. “Concurrent administration” as used herein thus includes administration of the agents at the same time or at different times. Administration of two or more agents at the same time can be achieved by a single formulation containing two or more active ingredients or by substantially simultaneous administration of two or more dosage forms with a single active agent.

The methods of the present invention are intended for use with any mammal that may experience the benefits of the methods of the invention. Foremost among such mammals are humans, although the invention is not intended to be so limited, and is applicable to veterinary uses. Thus, in accordance with the invention, “mammals” or “mammal in need” include humans as well as non-human mammals, particularly domesticated animals including, without limitation, cats, dogs, and horses.

### EXAMPLE 1

### 5-{4-[5-Butyl-2-oxo-3-(tetrahydro-pyran-4-yl)-imidazolidin-1-yl]-4′-methyl-[1,4′]bipiperidinyl-1′-carbonyl}-4,6-dimethyl-pyridine-2-carbonitrile (I-1, SCHEME A)

step 1—To a solution of A-1a (CASRN 685530-64-7, 25 g, 73.22 mmol) and THF (225 mL) was added dropwise MeMgBr (122 mL, 366 mmol, 3M in Et₂O). The solution was stirred at RT overnight, quenched with saturated NH₄Cl and diluted with EtOAc. The aqueous phase was extracted with EtOAc and the combined extracts washed sequentially with water and brine, dried (Na₂SO₄), filtered and evaporated to afford a white solid which was dried in vacuo to afford 23.85 g (99%) of A-1b.

step 2—The ketal A-1b (23.85 g) was dissolved in MEOH (225 mL), con HCl (20 mL) was added and the reaction mixture heated at reflux for 3 d. The solvent was evaporated and the residue dissolved in H₂O (150 mL) and 6M HCl was added (30 mL). The resulting solution was heated at reflux overnight. The reaction mixture was poured onto ice and the resulting solution made basic with 5M NaOH. The resulting solution was twice extracted with EtOAc and the combined extracts washed with H₂O, dried (Na₂SO₄), filtered and evaporated to afford A-2a as a golden oil.

step 3—To a solution of A-2a (2.71 g, 9.46 mmol) and (Boc)₂O (2.27 g, 10.41 mmol MeOH (80 mL) was added a suspension of 20% Pd(OH)₂/C (250 mg) and MeOH (20 mL). The reaction was twice evacuated and filled with H₂ then stirred at RT temperature for 22 h under an H₂ atmosphere maintained with a balloon. The reaction was filtered through CELITE® and the resulting filtrated concentrated in vacuo. The crude product was purified by SiO₂ chromatography eluting with an acetone/hexane gradient (0 to 20% acetone) to afford 0.592 g (21%) of A-2b.

step 4—To a solution of A-2b (0.604 g, 2.04 mmol), S(+)-2-amino-hexanol and DCE (10 mL) was added NaBH(OAc)₃ (0.562 g, 2.65 mmol) and DCE (5 mL). The resulting solution was stirred overnight at RT, quenched with 5M NaOH and the resulting mixture extracted with DCM. The aqueous phase was re-extracted with DCM and the combined extracts were washed with water, dried (Na₂SO₄), filtered and evaporated to afford A-3a as a thick syrup which was used without further purification.

The other enantiomer was prepared analogously from R-(−)-2-amino-hexanol.

step 5—To a mixture of A-3a (0.744 g, 1.87 mmol), K₂CO₃ (0.775 g, 5.61 mmol) and THF (10 mL) was added benzyl chloroformate and the resulting solution was stirred overnight at RT. The reaction was quenched with H₂O and diluted with EtOAc. The EtOAc solution was sequentially washed with 50% brine and brine, dried (Na₂SO₄), filtered and evaporated. The crude product was purified by SiO₂ chromatography eluting with a DCM/MeOH gradient (0-10% MEOH) to afford 0.388 g (39%) of A-3b. The major impurity isolated from the reaction mixture was spiro oxazolidine formed by intramolecular displacement of benzyl alcohol.

step 6—A solution of A-3b (0.388 g, 0.73 mmol), Ph₃P (0.211 g, 0.80 mmol), phthalimide (0.113 g, 0.77 mmol) and THF (10 mL) was cooled in an ice bath and DEAD (121 μL, 0.134 g, 0.77 mmol) was added via syringe. The solution was stirred at 0° C. for 15 min then warmed to RT for 2.5 d. The reaction mixture was concentrated to afford A-4 as a golden syrup which was used without further purification.

step 7—The phthalimide A4 was dissolved in EtOH (5 mL) and hydrazine hydrate (390 μL, 0.402 g, 8.03 mmol) was added. The solution was heated at reflux for 2 h, cooled, filtered and the filter cake was washed with EtOH. The filtrate was evaporated and the residue taken up in a minimal amount of warm DCM. The solution was cooled and filtered through a 4 μm filter and the resulting filtrate evaporated under reduced pressure to afford A-5a which was used without further purification.

step 8—To a solution of A-5a (0.194 g, 0.36 mmol) and tetrahydropyran-4-one (0.036 g, 0.36 mmol) in DCE (2 mL) was added NaBH(OAc)₃ (0.106 g, 0.50 mmol) and DCE (1 mL). The solution was stirred at RT overnight then quenched with 1M NaOH and extracted with DCM. The aqueous solution was extracted with DCM and the combined extracts were washed with brined and dried (Na₂SO4), filtered and evaporated. The product was purified by SiO₂ chromatography eluting with a MeOH/DCM gradient (0 to 10% MeOH). The recovered syrup was dried in vacuo 0.091 g (41%) of A-5b.

step 9—A solution of A-5b (0.091 g, 0.15 mmol), 20% Pd(OH)₂/C (9 mg) and MEOH (3 mL) and was vacuum purged with N₂ then maintained under an H₂ atmosphere under balloon pressure. The reaction mixture was stirred at RT overnight, filtered through CELITE® and the filtrate evaporated to afford a glassy solid which was dried in vacuo to afford 71 mg (100%) of A-6 which was used without further purification.

step 10—To a solution of A-6 (0.071 g, 0.15 mmol), pyridine (49 μL, 0.60 mmol) and DCM (3 mL) cooled to 0° C. was add slowly triphosgene. The solution was stirred at RT for 2.5 d under an inert atmosphere. The reaction was quenched by addition of saturated NaHCO₃ the resulting mixture extracted with DCM. The aqueous phase was extracted with DCM and the combined extracts were dried (Na₂SO₄), filtered and evaporated. The resulting syrup was dried in vacuo to afford A-7a which was used without further purification.

step 11—A solution of A-7a (0.076 g, 0.15 mmol), TFA (0.5 mL) and DCM (3 mL)was stirred at RT for 135 min, then evaporated and the residue partitioned between 5M KOH and EtOAc. The aqueous layer was extracted with EtOAc and the combined organic extracts washed with 50% brine, dried (Na₂SO₄), filtered and evaporated to afford A-7b as a glassy solid which was dried in vacuo then used without additional purification.

step 12—To a solution of A-7b (0.030 g, 0.074 mmol), 6-cyano-2,4-dimethyl-nicotinic acid (14 mg, 0.081 mmol), HOBt (13 mg, 0.096 mmol) and DMF (0.5 mL) was added a solution of EDCI (18 mg, 0.096 mmol) and DMF (0.5 mL) then DIPEA (39 μL, 0.222 mmol). The reaction mixture was stirred overnight at RT under an inert atmosphere. Mass spectrometry indicated about 30% conversion and a solution of additional carboxylic acid (14 mg), EDCI (18 mg, 0.096 mmol) and DMF (0.5 mL) was added. After the coupling reaction was complete by hplc/ms analysis, the reaction was quenched with H₂O and diluted with EtOAc. The aqueous phase was separated and thrice extracted with EtOAc. The combined EtOAc extracts were washed with brine, dried (Na₂SO₄), filtered and evaporated to afford an orange oil. The crude product was purified by SiO₂ chromatography eluting with a MeOH/DCM gradient (0 to 10% MeOH) to afford I-1

### EXAMPLE 2

### 4-Butyl-3-[1′-(4,6-dimethyl-pyriridine-5-carbonyl)-4′-methyl-[1,4′]bipiperidinyl-4-yl]-1-(tetrahydro-pyran-4-ylmethyl)-imidazolidin-2-one

step 1—To a solution of A-5a (0.194 g, 0.36 mmol) and 4-formyl-tetrahydropyran (0.041 g, 0.36 mmol) in DCE (2 mL) was added NaBH(OAc)₃ and additional DCE (1 mL). The reaction was stirred overnight at RT after which an additional 41 mg of aldehyde was added and stirring continued for another day. The reaction was quenched with 1N NaOH and diluted with DCM. The aqueous layer was separated and extracted with DCM. The combined DCM extracts were washed with brine, dried (Na₂SO₄), filtered and evaporated. The crude product was purified by SiO₂ chromatography eluting with MeOH/DCM (0 to 10% MeOH) and the recovered oil was concentrated in vacuo to afford 109 mg (48%) of A-5b (R′″=tetrahydropyran-4-ylmethyl).

The title compound was prepared from A-5b (R′″=tetrahydropyran-4-ylmethyl) as described in steps 9-12 of example 1 except in step 12, 4-6-dimethyl-pyrimidine-5-carboxylic acid was used in place of 6-cyano-2,4-dimethyl-nicotinic acid.

### EXAMPLE 3

### 1-(4,4-Difluoro-cyclohexylmethyl)-3-[1′-(2,4-dimethyl-pyridine-3-carbonyl)-4′-methyl-[1,4′]bipiperidinyl-4-yl]-imidazolidin-2-one

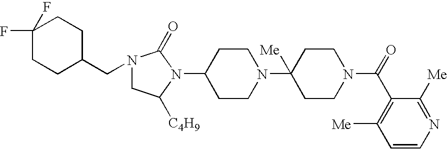

The title compound can be prepared by the procedures described in example 1 except in step 8, tetrahydropyran-4-one is replaced with 4,4-difluoro-cyclohexanecarboxaldehyde (CASRN 265108-36-9) and in step 12, 6-cyano-2,4-dimethyl-nicotinic acid is replaced with 2,4-dimethyl-nicotinic acid (CASRN 55314-30-2).

### EXAMPLE 4

### 1-[1′-(2,4-Dimethyl-pyridine-3-carbonyl)-4′-methyl-[1,4′]bipiperidinyl-4-yl]-3-(4-methoxy-cyclohexylmethyl)-imidazolidin-2-one

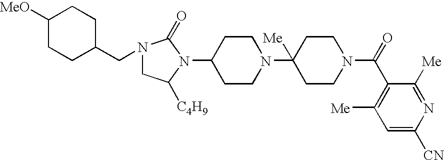

The title compound can be prepared by the procedures described in example 1 except in step 8, tetrahydropyran-4-one is replaced with 4-methoxy-cyclohexanecarboxaldehyde (CASRN 120552-57-0).

### EXAMPLE 5

### 5-{4-[3-(1-Acetyl-piperidin-4-ylmethyl)-2-oxo-imidazolidin-1-yl]-4′-methyl-[1,4′]bipiperidinyl-1′-carbonyl}-4,6-dimethyl-pyridine-2-carbonitrile

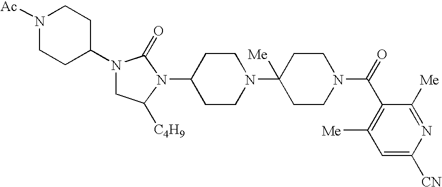

The title compound can be prepared by the procedures described in example 1 except in step 8, tetrahydropyran-4-one is replaced with 1-acetyl-piperidine-4-carbaldehyde (CASRN 155826-26-9).

### EXAMPLE 6

### 3-[1′-(4,6-Dimethyl-pyrimidine-5-carbonyl)-4′-methyl-[1,4′]bipiperidinyl-4-yl]-4-phenyl-1-(tetrahydro-pyran4-ylmethyl)-imidazolidin-2-one (I-6)

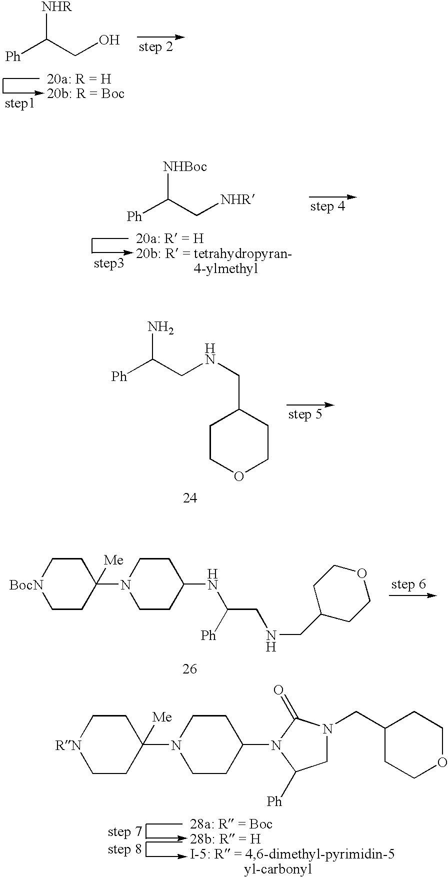

step 1—To a solution of 20a (4.42 g, 32.22 mmol), (Boc)₂O (7.74 g, 35.44 mmol) and Et₂O is added 1M NaOH (50 mL) and the resulting mixture is stirred overnight at RT. The two-phase suspension is filtered to remove insoluble material. The phases are separated and the aqueous layer is extracted with Et₂O and the combined ether extracts are washed with brine, dried (Na₂SO₄), filtered and concentrated in vacuo to afford a quantitative yield of 20b as a white solid.

step 2—To a solution of 20b (4.70 g, 19.81 mmol), phthalimide (3.06 g, 20.80 mmol), Ph3P (5.72 g, 21.79 mmol) and THF (150 mL) cooled to 0° C. was added DEAD (3.28 mL, 20.80 mmol) slowly via syringe. The resulting solution was stirred at 0° C. for 10 min, than warmed to RT for 5.5 h. The THF was evaporated the white residue was suspended in Et₂OH (150 mL) and hydrazine hydrate (10.90 g, 217.90 mmol) was added and the solution heated at reflux for 1.5 h, cooled and filtered. The filter cake was washed with EtOH and the combined EtOH solutions were evaporated. The residue taken up in DCM, filtered and the filtrate evaporated to afford a viscous yellow oil which was dried under high vacuum o afford 4.63 g (99%) of 22a which crystallized on standing and was used without further purification.

step 3—To a solution of 22a (0.380 g, 1.61 mmol), 4-formyl-tetrahydropyran (0.184 g, 1.61 mmol) and DCE (8 mL) was added a solution of NaBH(OAc)₃ and DCE (2 mL). The resulting solution was stirred overnight, quenched with 1M NaOH and the solution diluted with DCM. The phases were separated and the aqueous phase twice extracted with DCM. The combined organic extracts were dried (Na₂SO₄), filtered and concentrated in vacuo. The crude product was purified by SiO₂ chromatography elution in a MeOH/DCM gradient (0 to 10% MeOH to afford 0.188 g (ca 35%) of 22b containing a small amount of dialkylated product.

step 4—A solution of 22b (0.188 g, 0.56 mmol) and DCM (5 mL) was added TFA (0.50 mL) and stirred overnight at RT. The resulting solution was concentrated and the residue partitioned between EtOAc and 5M KOH. The organic phase was washed with brine, dried (Na₂SO₄), filtered and evaporated to yield 24 as a yellow syrup which was dried in vacuo and used without further purification.

step 5—To a solution of 24 (0.131 g, 0.56 mmol) and 4′-methyl-4-oxo-[1,4′]bipiperidinyl-1′-carboxylic acid tert-butyl ester (0.166 g, 0.56mmol) in DCE (3 mL) was added NaBH(OAc)₃ and DCE (2 mL) and the solution stirred for 5 d. The solution was quenched with 5 N NaOH and the resulting solution diluted with DCM. The phases were separated and the aqueous phase twice extracted with DCM. The combined DCM extracts were dried (Na₂SO₄), filtered and evaporated to afford 26 as a yellow oil which was used with further purification.

step 6—To a solution of 26(0.288 g, 0.56 mmol), pyridine (0.177 g, 0.56 mmol) and DCM (9 mL) cooled to 0° C. was slowly added triphosgene (0.166 g, 0.56 mmol). The solution was stirred at RT overnight then quenched with saturated NaHCO₃. The solution was diluted with DCM and he phases were separated. The aqueous phase was twice extracted with DCM and the combined DCM extracts were dried (Na₂SO₄), filtered and evaporated. The crude product was purified by SiO₂ chromatography eluting with a MeOH/DCM gradient (0 to 10% MeOH) to afford a clear glass which was dried in vacuo to afford 0.052 g (17%) of 28a.

step 7—A solution of 28a (0.052 g, 0.796 mmol), TFA (0.5 mL) and DCM (3 mL) was stirre at RT for 2 h. The solvents were evaporated and the residue dried in vacuo. The resulting product was dissolved in MeOH (20 mL) and MP-carbonate (0.200 g, macroporous triethylammonium methylpolystyrene carbonate) was added and the resulting mixture stirred overnight at RT. The pH was adjusted to about 8, the resin filtered, and the solvents evaporated in vacuo to afford 0.045 g of 28b which was used without further purification.

step 8—To a solution of 28b (0.045 g, 0.102 mmol), 4,6-dimethyl-pyrimidine-5-carboxylic acid (1.1 equivalents), HOBt (0.013 g, 0.1 mmol) in DMF (2 mL) was added sequentially EDCI (0.025 g, 0.13 mmol) and DIPEA (0.0387 g, 0.3 mmol). The resulting solution was stirred overnight at RT, concentrated in vacuo and purified by SiO₂ chromatography eluting with a MeOH/DCM gradient (5 to 10% MeOH) which yielded a glass which was triturated with Et₂O to 0.020 g of I-5.

### EXAMPLE 7

### (R)-3-[1′-(4,6-Dimethyl-pyrimidine-5-carbonyl)-4′-methyl-[1 ,4′]bipiperidinyl-4-yl]-4-phenyl-1-(tetrahydro-pyran-4-ylmethyl)-imidazolidin-2-one (1-5, SCHEME B)

step 1—R-phenylglycinol (B-1a, 19.4 g, 142 mmol)and BOC anhydride (32.5 g, 149 mmol) were combined in Eₜ₂O (300 mL) and 1M NaOH (100 mL) was added. The resulting suspension was stirred at 25° C. whereupon the mixture slowly became a two-phase solution. After 24 h a white precipitate had formed. The reaction was diluted with EtOAc and the phases were separated. The EtOAc was washed with water and brine, dried (Na₂SO₄), filtered, evaporated and dried in vacuo to afford 32.7 g (97%) B-1b as a white crystalline solid: ¹H NMR (CDCl₃, 300 MHz): δ 7.38-7.25 (m, 5H), 5.31 (broad s, 1H), 4.77 (broad s, 1H), 3.81 (m, 2H), 2.59 (broad s, 1H), 1.48 (s, 9H).

step 2—To a solution of B-2b (2.08 g, 8.77 mmol) and DCM (80 mL) cooled to 0° C. was added TEA (1.47 mL, 10.52 mmol) followed by mesyl chloride (0.74 mL, 9.65 mmol). The reaction was stirred at 0° C. for 1.5 h and at RT for 1 h. The reaction was diluted with DCM and washed successively with 1M HCl, 10% NaHCO₃ and brine. The organic phase was dried (Na₂SO₄), filtered and evaporated in vacuo to afford B-2a as a white powder which was used without further purification. ¹H NMR (CDCl₃, 300 MHz): δ 7.41-7.28 (m, 5H), 5.22 (d, 1H), 5.02 (broad s, 1H), 4.49-4.37 (m, 2H), 2.87 (s, 3H), 1.43 (s, 9H).

step 3—To a solution of B-2a (1.0 g, 3.17 mmol) and DMF (8 mL) was added sodium azide (289 mg, 4.44 mmol) and the mixture was stirred at 60° C. for 17 h, cooled and partitioned between water and ether. The phases were separated and the aqueous phase extracted with ether. The combined extracts were washed sequentially with water and brine, dried (Na₂SO₄), filtered and evaporated in vacuo to afford 557 mg of B-2b as a white solid: ¹H NMR (CDCl₃, 300 MHz): δ 7.44-7.28 (m, 5H), 5.30 (broad s, 1H), 4.88 (broad s, 1H), 3.67-3.63 (m, 2H), 1.44 (s, 9H).

step 4—To a solution of B-2b (200 mg, 0.76 mmol) in DCM (10mL) was added TFA (2 mL). The mixture was stirred at RT for 16 h, then concentrated in vacuo and partitioned between 1M NaOH and DCM. The phases were separated and the aqueous was extracted with DCM. The combined organic phases were washed with brine, dried (Na₂SO₄), filtered and evaporated to afford a clear oil. The crude material was combined with A-2b (225 mg, 0.76 mmol) in DCE (3 mL). To the solution was added 226 mg (1.06 mmol) of sodium triacetoxyborohydride and DCE (2 mL). The mixture was stirred at RT for 17 h then s quenched with 5M KOH and diluted with DCM. The phases were separated and the aqueous was extracted with DCM. The combined organic phases were washed with brine, dried (Na₂SO₄), filtered and evaporated to afford a light gold syrup. The crude material was purified by SiO₂ chromatography eluting with a DCM/MeOH gradient of (0 to 10% MeOH) to afford 255 mg (76%) of B-3a as a clear glass: MS (ESI) M+H=443.

step 5—To a solution of B-3a (255 mg, 0.58 mmol) in MeOH (5 mL) was added 15 mg of 5% Pd/C with MeOH (5 mL). The mixture was stirred at RT under balloon pressure of hydrogen for 17 h. The mixture was filtered through CELITE® and the filter cake washed with MeOH. The filtrate was stripped to afford 235 mg (98%) of B-3b as a white :MS (ESI) M+H=417.

step 6—To a solution of B-3b (117 mg, 0.28 mmol) and DCE (3 mL) was added 4-tetrahydropyran carboxaldehyde (32 mg, 0.28 mmol) followed by sodium triacetoxyborohydride (83 mg, 0.39 mmol) and DCE (2 mL). The reaction was stirred at RT for 17 h. The reaction was quenched with 5M KOH and diluted with DCM. The phases were separated and the aqueous was extracted twice with DCM. The combined organic phases were dried (Na₂SO₄), filtered and concentrated in vacuo to afford B-3c a light yellow syrup which was used without further purification: MS (ESI) M+H=515.

step 7—To a solution of B-3c (144 mg, 0.28 mmol) and pyridine (91 μL, 1.12 mmol) in DCM (3 mL) at 0° C. was added a solution of triphosgene (83 mg, 0.28 mmol) and DCM (1 mL). The reaction was stirred at 0° C. for 15 min and at RT for 17 h. The reaction was quenched with 10% NaHCO₃ and diluted with DCM. The phases were separated and the aqueous was extracted twice with DCM. The combined organic extracts were dried (Na₂SO₄), filtered and evaporated in vacuo to give a dark brown glass. The crude material was purified by SiO₂ chromatography eluting with a DCM/MeOH gradient (0 to 10% MeOH) to afford 0.120 g (79%) of the B-4a: MS (ESI) M+H=541.

step 8—To a solution of B-4a (120 mg, 0.22 mmol) in DCM (10 mL) was added TFA (1 mL). The mixture was stirred at RT for 16 h. The reaction was concentrated in vacuo and partitioned between 1M NaOH and DCM. The phases were separated and the aqueous was extracted twice with DCM. The combined organic phases were dried (Na₂SO₄), filtered and evaporated to afford 98 mg of B-4b as a yellow glass: MS (ESI) M+H=m/z 441.

step 9—To a solution of B4b (33 mg, 0.075 mmol) 4,6-dimethylpyriridine-5-carboxylic acid (11 mg, 0.075 mmol) and HOBt (13 mg, 0.098 mmol) in DMF (1 mL) was added EDAC (19 mg, 0.098 mmol) followed by DIPEA (29 mg, 0.23 mmol). The mixture was stirred at RT for 21 h. The reaction was quenched with water and diluted with DCM. The aqueous phase was extracted with DCM and the combined organic phases were dried (Na₂SO₄) and evaporated to a yellow oil. The crude material was purified by SiO₂ chromatography eluting with a DCM/MeOH (0 to 10% MeOH). The resulting off-white foam was dried to give 26 mg (60%) 1-5 of the title compound: MS (ESI) M+H=575 (M+H); [α]^{D}=44.6° (MeOH).

I-7 can be made be an analogously except in step 9, 4,6-dimethylpyrimidine-5-carboxylic acid is replaced with 6-cyano-2,4-dimethyl-pyridine-3-carboxylic acid.

I-8 can be made be an analogously except in step 9, 4,6-dimethylpyrimidine-5-carboxylic acid is replaced with 3-methyl-5-(trifluoromethyl)-4-isoxazolecarboxylic acid.

I-27 can be prepared using analogous procedures except in step 1, (R)-phenylglycinol is replaced with 3-fluoro-phenylglycinol.

### EXAMPLE 8

### (R)-3-[1′-(2,4-Dimethyl-pyridine-3-carbonyl)-4′-methyl-[1,4′]bipiperidinyl-4-yl]-1-(4-ethoxy-cyclohexylmethyl)-4-phenyl-imidazolidin-2-one (I-9)

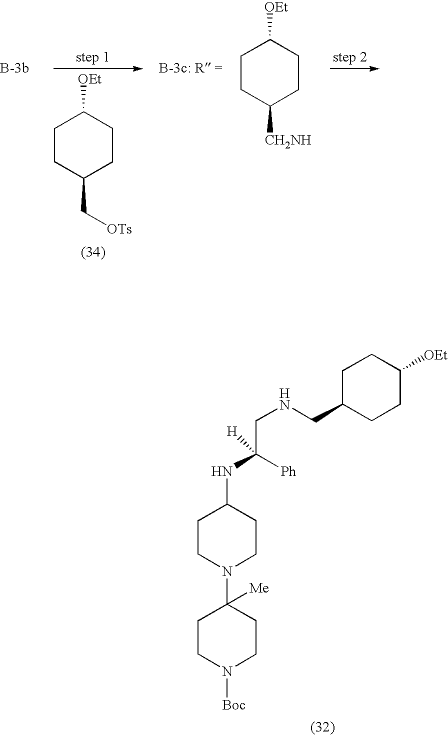

step 1—To a solution of B-3b (539 mg, 2.28 mmol) and MeCN (4 mL) was added NaHCO₃ (575 mg, 6.84 mmol) and a solution of 34 (0712 mg, 2.28 mmol) and MeCN (4 mL). The mixture was refluxed for 20 h then concentrated in vacuo. The residue was partitioned between water and DCM. The phases were separated and the aqueous was extracted with DCM. The combined organic extracts were washed with water and brine, dried (Na₂SO₄), filtered and concentrated in vacuo to a clear oil. The crude material was purified by SiO₂ chromatography eluting with a DCM/MeOH gradient (0 to 10% MeOH). The white crystalline material was dried to afford 304 mg (35%) of B-3c: ¹H NMR (CDCl₃, 300 MHz): δ 7.35-7.21 (m, 5H), 5.52 (d, 1H), 4.75 (broad s, 1H), 3.50 (q, 2H), 3.14 (m, 1H), 2.85 (m, 2H), 2.39 (d, 2H), 2.02 (bd, 2H), 1.79 (dt, 2H), 1.41 (bs, 9H&NH), 1.18 m, 5H), 0.89 (dq, 21H).

step 2—To a solution of B-3c (R″=trans-4-ethoxycyclohexyl-methylamine (304 mg, 0.81 mmol) and DCM (10 mL) was added TFA (1 mL). The mixture was stirred at RT for 16 h. The reaction was concentrated in vacuo and partitioned between 5M KOH and DCM The phases were separated and the aqueous was extracted with DCM. The combined organic phases were washed with water, dried (Na₂SO₄), and evaporated to a gold glass. The crude material was added to a solution of A-2b (240 mg, 0.81 mmol) and DCE (4 mL). To the solution was added a solution of sodium triacetoxyborohydride (240 mg, 1.13 mmol) and DCE (3 mL). The mixture was stirred at RT for 17 h. The reaction was quenched with 5M KOH and diluted with DCM. The phases were separated and the aqueous was extracted with DCM. The combined organic extracts were washed with brine, dried (Na₂SO₄), filtered and concentrated to afford a yellow syrup. The crude material was used without further purification: MS (ESI) M+H=557.

Cyclization of the imidazolidinone ring and introduction of the carboxamide was carried out as described in steps 7-9 of Example 7 to afford I-9.

I-10 can be made be an analogously except in step 9, 4,6-dimethylpyrimidine-5-carboxylic acid is replaced with 6-cyano-2,4-dimethyl-pyridine-3-carboxylic acid.

I-11 can be made be an analogously except in step 9, 4,6-dimethylpyrimidine-5-carboxylic acid is replaced with 3-methyl-5′-(trifluoromethyl)-4-isoxazolecarboxylic acid.

### EXAMPLE 9

### 3-[1′-(4,6-Dimethyl-pyrimidine-5-carbonyl)-4′-methyl-[1,4′]bipiperidinyl-4-yl]-4-phenyl-1-(tetrahydro-pyran-4-yl)-imidazolidin-2-one (I-12)

step) 1—To a solution of B-3b (325 mg, 0.78 mmol) and DCE (10 mL) was added tetrahydropyran-4-one (78 mg, 0.78 mmol) followed by a solution of sodium triacetoxyborohydride (231 mg, 1.09 mmol) and DCE (2 mL). The reaction was stirred at RT for 17 h. The reaction was quenched with 1M NaOH and diluted with DCM. The phases were separated and the aqueous was extracted twice with DCM. The combined organic extracts were dried (Na₂SO₄), filtered and concentrated in vacuo to afford 4′-methyl-4-[(R)-1-phenyl-2-(tetrahydro-pyran-4-ylamino)-ethylamino]-[1,4′]bipiperidinyl-1′-carboxylic acid tert-butyl ester (36) as a light yellow gum which was used without further purification: MS (ESI):M+K=501.

step 2—To a solution of the diamine from step 1(446 mg, 0.89 mmol), TEA (137 μL, 0.98 mmol) and DCE (13 mL) at 0° C. was added a solution of p-nitrophenyl chloroformate (197 mg, 0.98 mmol) and DCM (1 mL). The reaction was stirred at RT for 17 h. when additional TEA (68 μL, 0.49 mmol) and p-nitrophenyl chloroformate (99 mg, 0.49 mmol) were added and the reaction was stirred a further 21 h. The reaction was quenched with 2.5M NaOH and diluted with DCM. The phases were separated and the aqueous was extracted twice with DCM. The combined organic phases were dried (Na₂SO₄), filtered and concentrated in vacuo to afford a yellow gum. The crude material was purified by SiO₂ chromatography eluting with a DCM/MeOH gradient (0 to 10% MeOH). The light yellow glass was dried to afford 330 mg (71%) of 4′-methyl-4-[(R)-2-oxo-5-phenyl-3-(tetrahydropyran-4-yl)-imidazolidin-1-yl]-[1,4′]bipiperidinyl-1′-carboxylic acid tert-butyl ester (38): MS (ESI) M+H=527.

Cyclization of the imidazolidinone ring and introduction of the carboxamide was carried out as described in steps 7-9 of Example 7 except 4,6-dimethylpyrimidine-5-carboxylic acid is replaced with 6-cyano-2,4-dimethyl-pyridine-3-carboxylic acid to afford I-12.

I-13 can be made be an analogously except in step 9, 6-cyano-2,4-dimethyl-pyridine-3-carboxylic acid is replaced with 4,6-dimethylpyrimidine-5-carboxylic acid.

### EXAMPLE 10

### (R)-1-[1-(2,2-Difluoro-ethyl)-piperidin-4-ylmethyl]-3-[1′-(4,6-dimethyl-pyrimidine-5-carbonyl)-4′-methyl-[1,4′]bipiperidinyl-4-yl]-4-phenyl-imidazolidin-2-one (I-14, SCHEME C)

step 1—To a solution of B-3b (0.49 g, 0.00118 mol) and DCE (10 mL) was added C-1 (0.29 g, 0.00118 mol) followed by a solution of sodium triacetoxyborohydride (0.35 g, 0.00165 mol) and DME (2 mL). The reaction was stirred at RT for 17 h. The reaction was quenched with 5M KOH and diluted with DCM. The phases were separated and the aqueous layer was extracted twice with DCM. The combined organic phases were dried (Na₂SO₄), filtered and concentrated in vacuo to give the afford 0.76 g (100%) of C-2 as a light yellow syrup which was used without further purification.

step 2—To a solution of C-2 (0.76 g, 0.00117 mol) and pyridine (0.38 g, 0.0047 mol) in DCM (1 (15 mL) at 0° C. was added a solution of triphosgene (0.35 g, 0.00117 mol) triphosgene and DCM (1 mL). The reaction was stirred at 0° C. for 15 min and at RT for 17 h. The reaction was quenched with 10% NaHCO₃ and diluted with DCM. The phases were separated and the aqueous layer was extracted twice with DCM. The combined organic phases were dried (Na₂SO₄), filtered and concentrated in vacuo to afford a dark brown glass. The crude material was purified by SiO₂ chromatography eluting with a CH₂Cl₂/MeOH gradient (0 to 10% MeOH) to afford 0.62 g (79%) of C-3a as alight yellow glass after drying.

step 3—A mixture of C-3a (0.2 g, 0.00029 mol) Pd(OH)₂/C (100 mg) and MeOH (50 mL) in a flask was degassed, and filled with H₂ from a balloon. After 22 h the reaction mixture was filtered through CELITE and the cake was washed with MeOH. The solvent was removed in vacuo and the residue was dried under a high vacuum to afford 0.16 g (100%) of C-3b as a thick oil which solidified upon standing overnight.

step 4—To a suspension of NaHCO₃ (0.175 g, 0.0002 mol) in EtOAc-water mixture (1.25 mL/1.25 mL) was added 2,2′-difluoroethyl-trifluoromethyl sulfonate (0.076 g, 0.00035 mol) and the resulting mixture was warmed to 40° C. A solution of C-3b (0.16 g, 0.000297 mol) in EtOAc-water mixture (1.25 mL/1.25 mL) was added and stirring continued for 1 hour at 50° C. The phases were separated and the aqueous layer was extracted twice with EtOAc. The combined organic extracts were dried (MgSO₄), filtered and concentrated in vacuo to afford 0.16 g (89%) of C-3c (R″=CH₂CHF₂) as a glass.

step 5—A solution of C-3c (R″═CH₂CHF₂, 0.16 g, 0.000265 mol) in mixture of TFA 1 mL) and DCM (5 mL) was stirred for 1 h, concentrated in vacuo, and the residue was stirred in a mixture of 2 g of MP-carbonate (macroporous triethylammonium methylpolystyrene carbonate (0.5% inorganic antistatic agent) and MeOH (50 mL) for 8 h (The final pH of solution was 8). The resin was filtered, the solvent was removed in vacuo and crude (R)-1-[1-(2,2-Difluoro-ethyl)-piperidin-4-ylmethyl]-3-(4′-methyl-[1,4′]bipiperidinyl-4-yl)-4-phenyl-imidazolidin-2-one (40, 0.14 g) was use next step without further purification.

step 6—To a solution of 40 (38 mg, 0.075 mmol), HOBt (13 mg, 0.098 mmol) and DMF (1 mL) was added EDAC (19 mg, 0.098 mmol) followed by DIPEA (29 mg, 0.23 mmol). The mixture was stirred at RT for 21 h. The reaction was quenched with water and diluted with DCM. The phases were separated and the aqueous phase extracted with DCM. The combined organic extracts were dried (Na₂SO₄) and concentrated to a yellow oil. The crude material was purified by SiO₂ chromatography eluting with a DCM/MeOH gradient (0 to 10% MeOH) to afford 28 mg (60%) of I-14 as a white foam after drying: MS (ESI) M+H=638.

I-15 can be prepared analogously except in step 6, 4,6-dimethylpyrimidine-5-carboxylic acid is replaced with 6-cyano-2,4-dimethyl-pyridine-3-carboxylic acid.

### EXAMPLE 11

### 4-{(R)-3-[1′-(4,6-Dimethyl-pyrimidine-5-carbonyl)-4′-methyl-[1,4′]bipiperidinyl-4-yl]-2-oxo-4-phenyl-imidazolidin-1-ylmethyl}-piperidine-1-carboxylic acid methyl ester (I-16)

step 4—To a solution of C-3b (0.13 g, 0.00024 mol), TEA (67 μL, 0.0005 mol) and DCM (5 mL) was added methyl chloroformate (27 mg, 0.00029 mol) and the reaction stirred for 18 h at RT. The reaction mixture was diluted with DCM (10 mL), the phases separated and organic phase was washed with 1N HCl 1(10 mL). The organic phase was dried (MgSO₄), filtered and concentrated in vacuo to afford 0.12 g of C-3c (R″═C(═O)OMe) which was used without a further purification.

The compound from step 4 can be converted to 1-16 utilizing procedures analogous to those described in steps 5 and 6 of example 10.

I-17 can be prepared analogously except in step 6, 4,6-dimethylpyrimidine-5-carboxylic acid is replaced with 6-cyano-2,4-dimethyl-pyridine-3-carboxylic acid.

### EXAMPLE 12

### 5-{4-[(R)-3-(1-Methanesulfonyl-piperidin4-ylmethyl)-2-oxo-5-phenyl-imidazolidin-1-yl]-4′-methyl-[1,4′]bipiperidinyl-1′-carbonyl}-4,6-dimethyl-pyridine-2-carbonitrile (I-18 SCHEME C)

step 4—To a solution of C-3b (0.16 g. 0.000297 mol), TEA (75 μL, 0.00055 mol) and DCM (5 mL) was added MeSO₂Cl (25 μL, (0.00033 M) and the reaction mixture was stirred for 18 h at RT. The reaction mixture was, diluted with DCM (10 mL), the phases were separated and organic phase was washed with water (10 mL). The organic extract was dried (MgSO₄), filtered and concentrated in vacuo to afford 0.18 g of C-3c (R″=MeSO₂) which was used without a further purification.

step 5—A solution of C-3c (R″=MeSO₂, 0.18 g, 0.00029 mol) of in mixture of TFA (1 mL) and DCM (5 mL) was stirred for 1 h, concentrated at in vacuo, and the residue was stirred in a mixture of MP-carbonate (2 g) and MEOH (50 mL) for 8 h. The resin was filtered off, the solvent was removed in vacuo to afford 0.12 g of (R)-1-(1-methanesulfonyl-piperidin-4-ylmethyl)-3-(4′-methyl-[1,4′]bipiperidinyl-4-yl)-4-phenyl-imidazolidin-2-one which was used without further purification.

step 6—To a solution of (R)-1-(1-methanesulfonyl-piperidin-4-ylmethyl)-3-(4′-methyl-[1,4′]bipiperidinyl-4-yl)-4-phenyl-imidazolidin-2-one (39 mg, 0.075 mmol), 6-cyano-2,4-dimethyl-pyridine-3-carboxylic acid (13 mg, 0.075 mmol), HOBt (13 mg, 0.098 mmol) DMF (1 mL) was added EDAC (19 mg, 0.098 mmol) followed by DIPEA (29 mg, 0.23 mmol). The mixture was stirred at RT for 21 h. The reaction was quenched with water and diluted with DCM. The phases were separated phases and aqueous was extracted with DCM. The combined organic extracts were dried (Na₂SO₄), filtered and concentrated to a yellow oil. The crude material was purified by SiO₂ chromatography eluting with a DCM/MeOH gradient (0 to 10% MeOH) to afford 15 mg (30%) of I-18 after drying: MS (ESI): M+H=676.

I-19 can be made be an analogously except in step 9, 6-cyano-2,4-dimethyl-pyridine-3-carboxylic acid is replaced with 4,6-dimethylpyrimidine-5-carboxylic acid.

### EXAMPLE 13

### 5-{4-[(R)-3-(1-Acetyl-piperidin-4-ylmethyl)-2-oxo-5-phenyl-imidazolidin-1-yl]-4′-methyl-[1,4′]bipiperidinyl-1′-carbonyl}-4,6-dimethyl-pyridine-2-carbonitrile (1-20, SCHEME C)

step 4—To a solution of C-3b (0.16 g, 0.000297 mol), TEA (75 μL, 0.00055 mol) and DCM (5 mL) was added acetyl chloride (25 μL, 0.00033 M) and the resulting solution stirred for 18 h at RT. The reaction mixture was diluted with DCM (10 mL), the phases separated and organic layer was washed with water (10 mL). The combined organic extracts were dried (MgSO₄), filtered and concentrated in vacuo to afford 0.16 g of C-3c (R″=Ac) which was used without a further purification.

The compound from step 4 can be converted to I-20 utilizing procedures analogous to those described in steps 5 and 6 of example 12.

### EXAMPLE 14

### 4-{(R)-3-[1′-(6-Cyano-2,4-dimethyl-pyridine-3-carbonyl)-4′-methyl-[1,4′]bipiperidinyl-4-yl]-2-oxo-4-phenyl-imidazolidin-1-ylmethyl}-piperidine-1-carboxylic acid amide (I-21, SCHEME C)

step 4—To a solution of C-3b (0.1 g, 0.00018 mol) and DCM (3 mL) was added Me₃Si—NCO (0.42 mL, 0.0031 M) and solution stirred for 18 h at RT. The reaction mixture was diluted with DCM (10 mL), the phases separated and organic phase was washed with water (10 mL). The combined organic phase was dried (MgSO₄), filtered and concentrated in vacuo to afford 0.108 g (100%) of C-3c (R″═C(═O)NH₂) which was used without further purification.

The compound from step 4 can be converted to I-21 utilizing procedures analogous to those described in steps 5 and 6 of example 12.

I-22 is prepared analogously except in step 4, trimethylsilylisocyanate is replaced with methyl isocyanate and in step 6 (See example 12), 6-cyano-2,4-dimethyl-pyridine-3-carboxylic acid is replaced with 4,6-dimethylpyrimidine-5-carboxylic acid.

### EXAMPLE 15

### 5-{4-[(R)-3-(4-Hydroxy-cyclohexyl)-2-oxo-5-phenyl-imidazolidin-1-yl]-4′-methyl-[1,4′]bipiperidinyl-1′-carbonyl}-4,6-dimethyl-pyridine-2-carbonitrile (I-23 SCHEME C)

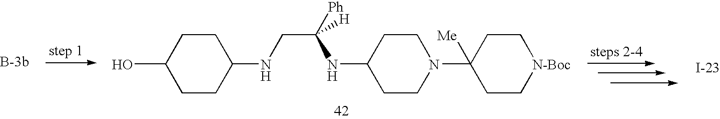

step 1—To a solution of C-3b (0.058 g, 0.000139 mol) and DCE (4 mL) was added 4-hydroxy-cyclohexanone (0.017 g, 0.00015 mol) followed by a solution of NaBH(OAc)₃ (0.041 g, 0.00019 mol) and DCE (2 mL). The reaction was stirred at RT for 17 h. The reaction was quenched with 5M KOH and diluted with DCM. The phases were separated and the aqueous layer was extracted twice with DCM. The combined organic phases were dried (Na₂SO₄), filtered and concentrated in vacuo to afford 0.076 g (100%) of 42 as a light yellow syrup which was used without further purification.

step 2—To a solution of 42 (0.6 g, 0.00117 mol), pyridine (0.38 g, 0.0047 mol) and DCM (15 mL) at 0° C. was added a solution of triphosgene (0.35 g, 0.00117 M) and DCM (1 mL). The reaction was stirred at 0° C. for 15 min and at RT for 17 h. The reaction was quenched with 10% NaHCO₃ and diluted with DCM. The phases were separated and the aqueous layer was extracted twice with DCM. The combined organic extracts were dried (Na₂SO₄), filtered and concentrated in vacuo to afford a dark brown glass. The crude material was purified by SiO₂ chromatography eluting with a DCM/MeOH gradient (0 to 10% MeOH) to afford 0.49 g (78%) of 4-[(R)-3-(4-hydroxy-cyclohexyl)-2-oxo-5-phenyl-imidazolidin-1-yl]-4′-methyl-[1,4′]bipiperidinyl-1′-carboxylic acid tert-butyl ester (44) as a light yellow glass.

step 3—A solution of 44 (0.031 g, 0.000057 mol), TFA (0.5 mL) and DCM (3 mL) was stirred for 1 h, concentrated under reduced pressure, and the residue was stirred with MP-carbonate (200 mg) and MeOH (20 mL) for 8 h. The resin was filtered and solvent removed in vacuo to afford 0.015 g (60%) of (R)-1-(4-hydroxy-cyclohexyl)-3-(4′-methyl-[1,4′]bipiperidinyl-4-yl)-4-phenyl-imidazolidin-2-one 46 which was used in next step without further purification.

step 4—To a solution of 46 (33 mg, 0.075 mmol), 6-cyano-2,4-dimethyl-pyridine-3-carboxylic acid (13 mg, 0.075 mmol), HOBt (13 mg, 0.098 mmol) and DMF (1 mL) were added EDAC (19 mg, 0.098 mmol) followed by DIPEA (29 mg, 0.23 mmol). The mixture was stirred at RT for 21 h. The reaction was quenched with water and diluted with DCM. The phases were separated and the aqueous phase was extracted with DCM. The combined organic extracts were dried (Na₂SO₄) and evaporated to a yellow oil. The crude material was purified by SiO₂chromatography eluting with a gradient of DCM and a solution of MeOH/DCM/NH₄OH (60:10:1) (95 to 70% DCM) to afford 20 mg (50%) of I-23.

### EXAMPLE 16

### (R)-1-(4-Hydroxy-cyclohexylmethyl)-3-[4′-methyl-1′-(3-methyl-5-trifluoromethyl-isoxazole-4-carbonyl)-[1,4′]bipiperidinyl-4-yl]-4-phenyl-imidazolidin-2-one (I-24)

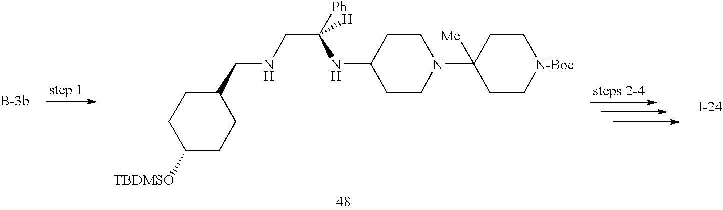

step 1—To a solution of C-3b 0.15 g (0.00036 mol) and DMF (4 mL) was added NaH (0.017 g (0.00072 M) followed by 4-(tert-butyl-dimethyl-silanyloxy)-cyclohexylmethyl toluenesulfonate (0.36 g, 0.0009 M). The reaction mixture was stirred at RT for 18 h, concentrated in vacuo, and purified by SiO₂ chromatography eluting with a DCM/MeOH gradient (0 to 10% MeOH) to afford 0.2 g (87%) of ((R)-2-{[4-(tert-butyl-dimethyl-silanyloxy)-cyclohexylmethyl]-amino}-1-phenyl-ethylamino)-4′-methyl-[1,4′]bipiperidinyl-1′-carboxylic acid tert-butyl ester.

step 2—To a solution of 48 (0.75 g, 0.00117 mol) and pyridine (0.38 g, 0.0047 mol) and DCM (15 mL) cooled to 0° C. was added a solution of triphosgene (0.35 g, 0.00117 mol) and DMF (1 mL). The reaction was stirred at 0° C. for 15 min and at RT for 17 h. The reaction was quenched with 10% NaHCO₃ and diluted with DCM. The phases were separated and the aqueous layer was extracted twice with DCM. The combined organic extracts were dried (Na₂SO₄), filtered and concentrated in vacuo to give a dark brown glass. The crude material was purified by SiO₂ chromatography eluting with a DCM/MeOH gradient (0 to 10% MeOH) to afford 0.61 g (78%) of 4-{(R)-3-[4-(tert-butyl-dimethyl-silanyloxy)-cyclohexylmethyl]-2-oxo-5-phenyl-imidazolidin-1-yl}-4′-methyl-[1,4′]bipiperidinyl-1′-carboxylic acid tert-butyl ester (50) after drying.

step 3—To a solution of 50 (0.15 g, 0.00022 mol), TFA (1 mL) and DCM (5 mL) was stirred for 1 h and concentrated in vacuo. The residue was stirred with MP-carbonate (200 mg) and MeOH (20 mL) for 8 h. The resin was filtered off, and the solvent was removed in vacuo to afford 0.070 g (70%) of (R)-1-(4-hydroxy-cyclohexylmethyl)-3-(4′-methyl-[1,4′]bipiperidinyl-4-yl)-4-phenyl-imidazolidin-2-one (52) which was used in next step without further purification.

step 4—To a solution of 52 (34 mg, 0.075 mmol), 3-methyl-5-trifluoromethyl-4,5-dihydro-isoxazole-4-carboxylic acid (15 mg, 0.075 mmol) and HOBt (13 mg, 0.098 mmol) and DMF (1 mL) were added sequentially EDAC (19 mg, 0.098 mmol) and DIPEA (29 mg, 0.23 mmol). The mixture was stirred at RT for 21 h. The reaction was quenched with water and extracted with DCM. The layers were separated and the aqueous phase extracted with DCM. The combined organic extracts were dried (Na₂SO₄) and concentrated to a yellow oil. The crude material was purified by SiO₂ chromatography eluting with a gradient of DCM and a solution of MeOH/DCM/NH₄OH (60:10:1) (95 to 70% DCM) to afford 22 mg (50%) of I-24 as a foam: MS (ESI) M+H=632(M+H).

### 4-(tert-butyl-dimethyl-silanyloxy)-cyclohexylmethyl toluenesulfonate

A solution of ethyl 4-hydroxy-cyclohexane carboxylate (5.0 g, 0.0236 mol), tert-butyl dimethyl silyl chloride (4.95 g), TEA (4.62 mL), DMAP (0.174 g) and DMF (52 mL) was stirred overnight at RT. The solution was concentrated in vacuo and the residue dissolved in EtOAc and washed sequentially with water and brine. The organic layer was dried (MgSO₄), filtered and evaporated to afford 8.8 g of the tosylate which was used without further purification.

To a solution of the ester from the previous step (8.86 g) and THF (100 mL) cooled to −20° C. was added dropwise a solution of LiAlH₄ and THF (37 mL, 1 M solution in THF) and after the addition the reaction mixture was allowed to warm to RT and stir overnight. The reaction was quenched with wet NaHSO3 and the resulting mixture stirred for 1 h. The resulting precipitate was filtered and the filtrate evaporated to afford 5.5 g (72%) of [4-(tert-butyl-dimethyl-silanyloxy)-cyclohexyl]-methanol.

To a solution of [4-(tert-butyl-dimethyl-silanyloxy) -cyclohexyl]-methanol (5.5 g, 0.0225 mol) in pyridine (20 mL) cooled to 0° C. was added p-toluenesulfonyl chloride (4.7 g, 0.0248 mol). The reaction was stirred overnight at RT, poured into ice water and extracted with DCM. The organic phase was washed sequentially with water and brine, dried (MgSO₄), filtered and evaporated. The cis and trans isomers were separated by SiO₂ chromatography eluting with 2% EtOAc in hexane.

I-25 can be prepared using analogous procedures except in step 1, by 4-(tert-butyl-dimethyl-silanyloxy)-cyclohexylmethyl benzenesulfonate is replaced with tetrahydro-furan-3-ylmethyl ester toluenesulfonate and in step 4, 3-methyl-5-trifluoromethyl-4,5-dihydro-isoxazole-4-carboxylic acid is replaced with 6-cyano-2,4-dimethyl-pyridine-3-carboxylic acid.

### Toluene-4-sulfonic acid tetrahydro-furan-3-ylmethyl ester

To a solution of 5 g (48.9 mmol) of tetrahydro-3-furanomethanol in pyridine (30 mL) cooled to 0° C. was added a solution of p-toluenesulfonyl chloride (9.3 g, 48.9 mmol) and DCM (30 mL) and the resulting solution was stirred at RT for 12 h. The reaction mixture was poured onto a mixture of 6 N HCl and ice. The aqueous phase was thrice extracted with DCM and the combined organic extracts were washed with water and brine, dried (Na₂SO₄) and evaporated to afford 9.5 g of the title compound as an oil. The enantiomers were separated by chiral HPLC to afford the two enantiomers with an enantiomeric excess of >95%

### EXAMPLE 17

### 5-{4-[(S)-3-(4-Ethoxy-cyclohexylmethyl)-2-oxo-5-pyridin-2-yl-imidazolidin-1-yl]-4′-methyl-[1,4′]bipiperidinyl-1′-carbonyl}-4,6-dimethyl-pyridine-2-carbonitrile (I-26)

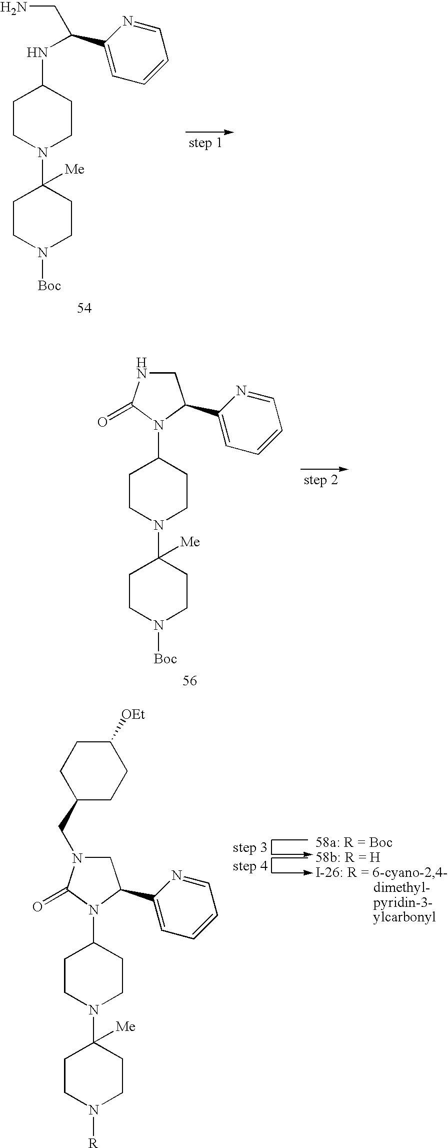

The diamine 54 can be prepared from (R)-β-amino-2-pyridineethanol (CASRN 160821-26-1) utilizing procedures analogous to steps I to 5 of example 7.

step 1—To a solution of 54 (102 mg, 0.24 mmol), DIPEA (51 μL, 0.29 mmol) and DCM (4 mL) at RT was added in one portion p-nitrophenyl chloroformate (55 mg, 0.26 mmol). The reaction was stirred at RT for 18 h then quenched with 2.5M NaOH and diluted with DCM. The phases were separated and the aqueous was extracted twice with DCM. The combined organic extracts were dried (Na₂SO₄), filtered and concentrated in vacuo. The crude material was purified by SiO₂ chromatography eluting with a DCM/MeOH gradient (0 to 10% MeOH) to afford 81 mg (75%) of 56: MS (ESI): M+H=444.3.

step 2—A mixture of 56 (81 mg, 0.18 mmol), NaOH (30 mg, 0.73 mmol), tetrabutyl ammonium bromide (3 mg, 0.09 mmol), K₂CO₃ (38 mg, 0.27 mmol) and trans-4-ethoxy-cyclohexylmethyl toluensulfonate (86 mg, 0.27 mmol) and toluene (4 mL) was heated at 90° C. for 18 h. Water was added, the phases were separated and the aqueous was extracted with DCM. The combined organic extracts were dried (MgSO₄) and concentrated. The crude material was purified by SiO₂ chromatography eluting with a DCM/MeOH gradient (0 to 10% MeOH) to afford 88 mg (83%) of the 58a.

Steps 3 and 4 can be carried out utilizing procedures analogous to steps 8 and 9 of example 7 except in the final step 4,6-dimethylpyrimidine-5-carboxylic acid is replaced with 6-cyano-2,4-dimethyl-pyridine-3-carboxylic acid to afford 58c.

### EXAMPLE 18

### Human CCR5 Receptor-Ligand Binding Assay Protocol

Human CCR5 receptor (Genebank ID: 29169292) was cloned into mammalian expression vector, pTarget (Promega). The construct was transfected into CHO-G_{α16} cells by using Fugene Reagent (Roche). Clones were selected under antibiotic pressure (G418 and Hygromycin) and sorted 4 times with a fluorescence activates cell sorter and a monoclonal antibody specific for CCR5 receptor (BD Biosciences Pharmigen, Mab 2D7, Cat. No. 555993). The clone with highest expression (100,000 copies per cell) was chosen for the binding assays.

Adherent cells in 225 mL tissue culture flask (˜90% confluent) were harvested using 1 mM EDTA in PBS (phosphate-buffered saline) without Ca²⁺ and Mg²⁺. Cells were washed twice with PBS containing no Ca²⁺ and Mg²⁺. CHO-G_{α16}-hCCR5 cells were then resuspended (1×10⁶/mL) in ice cold binding buffer (50 mM HEPES, 1 mM CaCl₂, 5 mM MgCl₂, 0.5% BSA, 0.05% NaN₃, pH 7.24), pH 7.4), supplemented with freshly made 0.5% BSA and 0.05% NaN₃.

Eighty μl CHO-G_{α16}-hCCR5 (1×10⁶/mL) cells were added to 96 well plates. All dilutions were made in binding buffer (50 mM HEPES, 1 mM CaCl₂, 5 mM MgCl₂, 0.5% BSA, 0.05% NaN₃, pH 7.24).

The plates were incubated on a cell shaker at RT for 2 h with a final concentration of 0.1 nM ¹²⁵I RANTES or ¹²⁵I MIP-1α or ¹²⁵I MIP-1β. The compound dilutions were made in PBS, 1% BSA. Total reaction volume was 100 μl per well. The test compounds were added to the cells prior to the addition of radioligand.

After incubation, the cells were harvested onto GF/C filter plates using Packard cell harvester. Filters were pretreated with 0.3% PEI/0.2% BSA for 30 min. The filter plate was washed rapidly 5 times with 25 mM HEPES, 500 mM NaCl, 1 mM CaCl₂ and 5 mM MgCl₂ adjusted to pH 7.1. Plates were dried in oven (70° C.) for 20 min, added with 40 μl scintillation fluid and sealed with Packard TopSeal-A. Packard Top Count was used to measure of the radioactivity for 1 min per well.

Total binding was determined with control wells added with radioisotope and buffer and the non-specific binding was determined using an excess cold RANTES to some of the control wells. Specific binding was determined by subtracting the non-specific form total binding. Results are expressed as the percentage of specific ¹²⁵I RANTES binding. IC₅₀ values were determined using varying concentrations of the test ligand in triplicates and the data was analyzed using GraphPad Prism (GraphPad, San Diego, Calif.). c,

### EXAMPLE 19

### CCR5-Mediated CCF Assay

CCF assay was performed as described before (C. Ji, J. Zhang, N. Cammack and S. Sankuratri, J. Biomol. Screen. 2006 11(6):652-663). Hela-R5 cells (express gp160 from R5-tropic virus and HIV-1 Tat) were plated in 384 well white culture plates (BD Bioscience, Palo Alto, Calif.) at 7.5×10³ cells per well in phenol red-free Dulbecco's Modified Eagle Medium (DMEM) supplemented with 10% FBS, 1× Pen-Strep, 300 μg/mL G418, 100 μg/mL hygromycin, and 1 μg/mL Doxycycline (Dox) (BD Bioscience, Palo Alto, Calif.), using Multimek (Beckman, Fullerton, Calif.) and incubated at 37° C. overnight to induce the expression of gp160. Ten μL diluted compounds in medium containing 5% DMSO were added to the cells, followed by the addition of CEM-NKr-CCR5-Luc (obtained from NIH AIDS Research & Reference Reagents Program) that expresses CD4 and CCR5 and carries a HIV-2 long terminal repeat (LTR)-driven luciferase reporter gene at 1.5×10⁴ cells/15 μL/well and incubated for 24 hrs. At the end of co-culture, 15 μL of Steady-Glo luciferase substrate was added into each well, and the cultures were sealed and gently shaken for 45 min. The luciferase activity were measured for 10 sec per well as luminescence by using 16-channel TopCount NXT (PerkinElmer, Shelton, Conn.) with 10 min dark adaptation and the readout is count per second (CPS). For the drug interaction experiments, small molecule compounds or antibodies were serially diluted in serum-free and phenol red-free RPMI containing 5% DMSO (CalBiochem, La Jolla, Calif.) and 1× Pen-Strep Five μL each of the two diluted compound or mAb to be tested for drug-drug interactions were added to the Hela-R5 cells right before the addition of target cells.

TABLE V

| | Cell-Cell Fusion (CCF) Assay |
|---|---|
| Cpd. No. | IC₅₀ (μM) |
| I-1 | 0.0085 |

### EXAMPLE 20

### Antiviral Assay

The sensitivity of a recombinant HIV-1 virus pseudotyped with the envelope proteins of the CCR5-tropic virus NLBal to test compounds was determined in a Luciferase reporter assay using JC53BL cells. NLBal pseudotyped HIV-1 was generated by calcium phosphate transfection of 293T cells (ATCC) with equal amounts of DNA of an envelope-deleted HIV-1 plasmid and of a NLBal envelope expression plasmid. The media (DMEM, 10% fetal bovine serum, 1% Penicillin/streptomycin, 1% Glutamine, all Gibco) was changed 16 h post-transfection and virus containing supernatant was harvested 48 h post-transfection. To determine the sensitivity of NLBal pseudotyped HIV-1, 25.000 JC53BL cells (NIH AIDS Reagent Program) were infected with NLBal pseudotyped HIV-1 in presence of a drug gradient in white 96 well plates (Greiner Bio-one). The volume was adjusted to 200 μL using assay media (DMEM, 10% fetal bovine serum, 1% Penicillin/streptomycin, 1% Glutamine). After incubation at 37° C., 90% relative humidity, 5% CO₂ for 3 days, 50 μL of Steady-Glo® Luciferase reagent (Promega) was added, incubated for 5 min at RT and luminescence was measured using a luminometer (Luminoskan, Thermo). The 50% and 90% inhibitory concentrations were calculated using Microsoft XL Fit 4 software.

TABLE VI

| | Antiviral Assay |
|---|---|
| Cpd. No. | IC₅₀ (μM) |
| I-1 | 0.0198 |

### EXAMPLE 21

Pharmaceutical compositions of the subject Compounds for administration via several routes were prepared as described in this Example.

Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation (D)

| Ingredient | % wt./wt. |
|---|---|
| Active ingredient | 0.25 g |
| Sodium Chloride | qs to make isotonic |
| Water for injection to | 100 ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

Suppository Formulation (E)

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

Topical Formulation (F)

| Ingredients | grams |
|---|---|
| Active compound | 0.2-2 |
| Span 60 | 2 |
| Tween 60 | 2 |
| Mineral oil | 5 |
| Petrolatum | 10 |
| Methyl paraben | 0.15 |
| Propyl paraben | 0.05 |
| BHA (butylated hydroxy anisole) | 0.01 |
| Water | q.s. 100 |

All of the ingredients, except water, are combined and heated to about 60° C. with stirring. A sufficient quantity of water at about 60° C. is then added with vigorous stirring to emulsify the ingredients, and water then added q.s. about 100 g.

### Nasal Spray Formulations (G)

Several aqueous suspensions containing from about 0.025-0.5 percent active compound are prepared as nasal spray formulations. The formulations optionally contain inactive ingredients such as, for example, microcrystalline cellulose, sodium carboxymethylcellulose, dextrose, and the like. Hydrochloric acid may be added to adjust pH. The nasal spray formulations may be delivered via a nasal spray metered pump typically delivering about 50-100 microliters of formulation per actuation. A typical dosing schedule is 24 sprays every 4-12 hours.

The features disclosed in the foregoing description, or the following claims, expressed in their specific forms or in terms of a means for performing the disclosed finction, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

We claim:

1. A compound according to formula I wherein 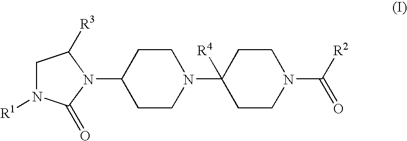
R¹ is: (a) C₃₋₆ cycloalkyl wherein said cycloalkyl is optionally substituted with one to three groups independently selected from the group consisting of hydroxy, C₁₋₃ allyl, oxo, halogen and C₁₋₆ alkoxy wherein any carbon atom adjacent only to other carbon atoms can be replaced by an oxygen atom;
(b) C₃₋₆ cycloalkyl-C₁₋₃ alkyl wherein said cycloalkyl is optionally substituted with one to three groups independently selected from the group consisting of hydroxy, C₁₋₃ alkyl, oxo, halogen and C₁₋₆ alkoxy wherein any carbon atom adjacent only to other carbon atoms can be replaced by an oxygen atom;
(c) tetrahydropyranyl, tetrahydropyranylmethyl, tetrahydrofuranyl, tetrahydrofuranylmethyl, or [1,4]dioxanyl; 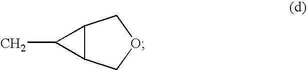 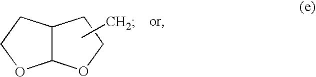 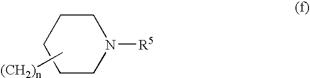
wherein R⁵ is C₁₋₆ acyl, C₁₋₆ alkoxycarbonyl, C₁₋₆ alkyl-SO₂, C₁₋₆ haloalkyl, C₃₋₆ cycloalkyl, carbamoyl, C₁₋₃ alkylcarbamoyl, tetrahydrofuranyl or tetrahydropyranyl and n is 1-3;
R³ is C₁₋₆ alkyl, pyridinyl or phenyl optionally substituted with 1 to 3 halogens
R⁴ is hydrogen or C₁₋₃ alkyl;
R² is selected from the group consisting of (a) to (e) and (f):
(a) 4,6-dimethyl-pyrimidin-5-yl;
(b) 2,4-dimethyl-pyridin-3-yl;
(c) 2,4-dimethyl-1-oxy-pyridin-3-yl
(d) 6-cyano-2,4-dimethyl-pridin-3-yl;
(e) 2,4-dimethyl-6-oxo-1,6-dihydro-pyridin-3-yl;
(f) 4,6-dimethyl-2-trifluoromethyl-pyrimidin-5-yl; or,
(g) 3-methyl-5-trifluoromethyl-isoxazol-4-yl or
a pharmaceutically acceptable acid addition salt thereof.

2. A compound according to claim 1 wherein R³ is C₃₋₅ alkyl and R⁴ is methyl.

3. A compound according to claim 1 wherein R³ is optionally substituted phenyl or pyridinyl and R⁴ is methyl.

4. A compound according to claim 2 wherein R¹ is tetrahydropyranyl or tetrahydropyranylmethyl and R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl.

5. A compound according to claim 2 wherein R¹ is 4-(C₁₋₃ alkoxy)-cyclohexyl-methyl, 4,4-difluorocyclohexyl-methyl, 4-oxo-cyclohexyl-methyl or 4-hydroxycyclohexyl-methyl and R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl.

6. A compound according to claim 2 wherein R¹ is 4-(C₁₋₃ alkoxy)-cyclohexyl, 4,4-difluorocyclohexyl, 4-oxo-cyclohexyl or 4-hydroxycyclohexyl and R² is 4,6-dimethyl-pyrimidin-5-yl, 2,4-dimethyl-pyridin-3-yl, 3-methyl-5-trifluoromethyl-isoxazol-4-yl or 6-cyano-2,4-dimethyl-pridin-3-yl.

7. A compound according to claim 2 wherein R¹ is (i): 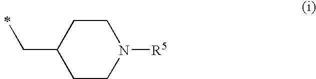
and R⁵ is C₁₋₃ acyl, C₁₋₃ alkylsulfonyl, C₁₋₃ haloalkyl or C₁₋₃ alkyl.

8. A compound according to claim 2 wherein R¹ is (ii):

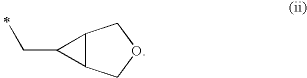

9. A compound according to claim 3 wherein R¹ is tetrahydropyranyl, tetrahydropyranylmethyl or tetrahydrofuranylmethyl and R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl.

10. A compound according to claim 3 wherein R¹ is 4-(C₁₋₃ alkoxy)-cyclohexyl-methyl, 4,4-difluorocyclohexyl-methyl, 4-oxo-cyclohexyl-methyl or 4-hydroxycyclohexyl-methyl and R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl.

11. A compound according to claim 3 wherein R¹ is 4-(C₁₋₃ alkoxy)-cyclohexyl, 4,4-difluorocyclohexyl, 4-oxo-cyclohexyl or 4-hydroxycyclohexyl and R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl.

12. A compound according to claim 3 wherein R¹ is (i): 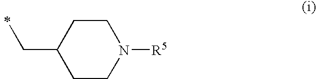
and R⁵ is C₁₋₃ acyl, C₁₋₃ alkylsulfonyl, C₁₋₃ haloalkyl or C₁₋₃ alkyl.

13. A compound according to claim 12 wherein R² is 4,6-dimethyl-pyrimidin-5-yl, 3-methyl-5-trifluoromethyl-isoxazol-4-yl, 2,4-dimethyl-pyridin-3-yl or 6-cyano-2,4-dimethyl-pridin-3-yl.

14. A compound according to claim 3 wherein R¹ is (ii):

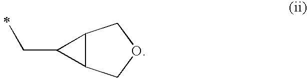

15. A compound according to formula I selected from the group consisting of:
5-{4-[5-butyl-2-oxo-3-(tetrahydro-pyran-4-yl)-imidazolidin-1-yl]-4′-methyl-[1,4′bipiperidinyl-1′-carbonyl}-4,6-dimethyl-pyridine-2-carbonitrile;
4-butyl-3-[1′-(4,6-dimethyl-pyrimidine-5-carbonyl)-4′-methyl-[1,4′]bipiperidinyl-4-yl]-1-(tetrahydro-pyran-4-yl)-imidazolidin-2-one;
4-butyl-3-[1′-(4,6-dimethyl-pyrimidine-5-carbonyl)-4′-methyl-[1,4′]bipiperidinyl-4-yl]-1-(tetrahydro-pyran-4-ylmethyl)-imidazolidin-2-one;
5-{4-[5-Butyl-2-oxo-3-(tetrahydro-pyran-4-ylmethyl)-imidazolidin-1-yl]-4′-methyl-[1,4′]bipiperidinyl-1′-carbonyl}-4,6-dimethyl-pyridine-2-carbonitrile;
(R)-3-[1′-(4,6-dimethyl-pyrimidine-5-carbonyl)-4′-methyl-[1,4′]bipiperidinyl-4-yl]-4-phenyl-1-(tetrahydro-pyran-4-ylmethyl)-imidazolidin-2-one;
3-[1′-(4,6-dimethyl-pyrimidine-5-carbonyl)-4′-methyl-[1,4′]bipiperidinyl-4-yl]-4-phenyl-1-(tetrahydro-pyran-4-ylmethyl)-imidazolidin-2-one;
4,6-dimethyl-5-{4′-methyl-4-[(R)-2-oxo-5-phenyl-3-(tetrahydro-pyran-4-ylmethyl)-imidazolidin-1-yl]-[1,4′]bipiperidinyl-1′-carbonyl}-pyridine-2-carbonitrile;
(R)-3-[4′-methyl-1′-(3-methyl-5-trifluoromethyl-isoxazole-4-carbonyl)-[1,4′]bipiperidinyl-4-yl]-4-phenyl-1-(tetrahydro-pyran-4-ylmethyl)-imidazolidin-2-one;
(R)-3-[1′-(4,6-dimethyl-pyrimidine-5-carbonyl)-4′-methyl-[1,4′]bipiperidinyl-4-yl]-1-(4-ethoxy-cyclohexylmethyl)-4-phenyl-imidazolidin-2-one;
5-{4-[(R)-3-(4-ethoxy-cyclohexylmethyl)-2-oxo-5-phenyl-imidazolidin-1-yl]-4′-methyl-[1,4′]bipiperidinyl-1′-carbonyl}-4,6-dimethyl-pyridine-2-carbonitrile;
(R)-1-(4-ethoxy-cyclohexylmethyl)-3-[4′-methyl-1′-(3-methyl-5-trifluoromethyl-isoxazole-4-carbonyl)-[1,4′]bipiperidinyl-4-yl]-4-phenyl-imidazolidin-2-one;
4,6-dimethyl-5-{4′-methyl-4-[2-oxo-5-phenyl-3-(tetrahydro-pyran-4-yl)-imidazolidin-1-yl]-[1,4′]bipiperidinyl-1′-carbonyl}-pyridine-2-carbonitrile;
3-[1′-(4,6-dimethyl-pyrimidine-5-carbonyl)-4′-methyl-[1,4′]bipiperidinyl-4-yl]-4-phenyl-1-(tetrahydro-pyran-4-yl)-imidazolidin-2-one;
(R)-1-[1-(2,2-difluoro-ethyl)-piperidin-4-ylmethyl]-3-[1′-(4,6-dimethyl-pyrimidine-5-carbonyl)-4′-methyl-[1,4′]bipiperidinyl-4-yl]4-phenyl-imidazolidin-2-one;
5-(4-{(R)-3-[1-(2,2-difluoro-ethyl)-piperidin-4-ylmethyl]-2-oxo-5-phenyl-imidazolidin-1-yl}-4′-methyl-[1,4′]bipiperidinyl-1′-carbonyl)-4,6-dimethyl-pyridine-2-carbonitrile;
4-{(R)-3-[1′-(4,6-dimethyl-pyrimidine-5-carbonyl)-4′-methyl-[1,4′]bipiperidinyl-4-yl]-2-oxo-4-phenyl-imidazolidin-1-ylmethyl}-piperidine-1-carboxylic acid methyl ester;
4-{(R)-3-[1′-(6-cyano-2,4-dimethyl-pyridine-3-carbonyl)-4′-methyl-[1,4′]bipiperidinyl-4-yl]-2-oxo-4-phenyl-imidazolidin-1-ylmethyl}-piperidine-1-carboxylic acid methyl ester;
5-{4-[(R)-3-(1-methanesulfonyl-piperidin-4-ylmethyl)-2-oxo-5-phenyl-imidazolidin-1-yl]-4′-methyl-[1,4′]bipiperidinyl-1′-carbonyl}-4,6-dimethyl-pyridine-2-carbonitrile;
4-(9-cyclopentyl-7,7-difluoro-5-methyl-6-oxo-6,7,8,9-tetrahydro-5H-pyrimido[4,5-b][1,4]diazepin-2-ylaniino)-N-(4-hydroxy-butyl)-3-methoxy-benzamide
5-{4-[(R)-3-(1-acetyl-piperidin-4-ylmethyl)-2-oxo-5-phenyl-imidazolidin-1-yl]-4′-methyl-[1,4′]bipiperidinyl-1′-carbonyl}-4,6-dimethyl-pyridine-2-carbonitrile;
4-{(R)-3-[1′-(2,4-dimethyl-pyridine-3-carbonyl)-4′-methyl-[1,4′]bipiperidinyl-4-yl]-2-oxo-4-phenyl-imidazolidin-1-ylmethyl}-piperidine-1-carboxylic acid amide;
4-{(R)-3-[1′-(4,6-dimethyl-pyrimidine-5-carbonyl)-4′-methyl-[1,4′]bipiperidinyl-4-yl]-2-oxo-4-phenyl-imidazolidin-1-ylmethyl}-piperidine-1-carboxylic acid methylamide;
5-{4-[(R)-3-(4-hydroxy-cyclohexyl)-2-oxo-5-phenyl-imidazolidin-1-yl]-4′-methyl-[1,4′]bipiperidinyl-1′-carbonyl}-4,6-dimethyl-pyridine-2-carbonitrile;
(R)-1-(4-hydroxy-cyclohexylmethyl)-3-[4′-methyl-1′-(3-methyl-5-trifluoromethyl-isoxazole-4-carbonyl)-[1,4′]bipiperidinyl-4-yl]-4-phenyl-imidazolidin-2-one;
4,6-dimethyl-5-(4′-methyl-4-{(R)-2-oxo-5-phenyl-3-[(S)-1-(tetrahydro-furan-3-yl)methyl]-imidazolidin-1-yl}-[1,4′]bipiperidinyl-1′-carbonyl)-pyridine-2-carbonitrile; compound with formic acid;
5-{4-[(S)-3-(4-ethoxy-cyclohexylmethyl)-2-oxo-5-pyridin-2-yl-imidazolidin-1-yl]-4′-methyl-[1,4′]bipiperidinyl-1′-carbonyl}-4,6-dimethyl-pyridine-2-carbonitrile; and
3-[1′-(4,6-dimethyl-pyrimidine-5-carbonyl)-4′-methyl-[1,4′]bipiperidinyl-4-yl]-4-(3-fluoro-phenyl)-1-(tetrahydro-pyran-4-ylmethyl)-imidazolidin-2-one; or,
pharmaceutically acceptable salts thereof.

16. A method for treating or preventing an human immunodeficiency virus (HIV-1) infection, or treating AIDS or ARC, in a patient in need thereof which comprises administering to the patient in need thereof a therapeutically effective amount of a compound according to claim 1.

17. A method according to claim 16 further comprising co-administering a therapeutically effective amount of one or more inhibitors selected from the group consisting of HIV-1 nucleoside reverse transcriptase inhibitors, non-nucleoside reverse transcriptase inhibitors, HIV-1 protease inhibitors, an integrase inhibitor and a HIV-1 viral fusion inhibitors and a compound of claim 1.

18. A method for treating rheumatoid arthritis comprising administering a therapeutically effective amount of a compound of according to claim 1 to a patient in need thereof.

19. A method according to claim 18 further comprising co-administering a therapeutically effective amount of one or more anti-inflammatory or analgesic compounds and a compound of claim 1.

20. A method for treating asthma or congestive obstructive pulmonary disease (COPD) comprising administering a therapeutically effective amount of a compound according to claim 1 to a patient in need thereof.

21. A method for treating solid organ transplant rejection comprising administering a therapeutically effective amount of a compound according to claim 1 to a patient in need.

22. A method according to claim 21 further comprising co-administering a therapeutically effective amount of one or more anti-rejection drugs or immunomodulators and a compound of claim 1.

23. A pharmaceutical composition comprising a compound according to claim 1 and at least one pharmaceutically acceptable carrier, diluent or excipient.
